(19) United States
(12) Patent Application Publication
(10) Pub. No.: US 2020/0221936 A1
(43) Pub. Date: Jul. 16, 2020

(54) Measurement Device

(71) Applicants: Muruga Raj, Ardsley, NY (US); Sanjeev Kumar, Greater Noida (IN)

(72) Inventors: Muruga Raj, Ardsley, NY (US); Sanjeev Kumar, Greater Noida (IN)

(21) Appl. No.: 16/830,166

(22) Filed: Mar. 25, 2020

Related U.S. Application Data
(62) Division of application No. 15/338,408, filed on Oct. 30, 2016.
(60) Provisional application No. 62/290,440, filed on Feb. 2, 2016.
(60) Provisional application No. 62/287,005, filed on Jan. 26, 2016.

(51) Int. Cl. A61B 1/00; A61B 1/015

(52) U.S. Cl. CPC A61B 1/00137; A61B 1/015; A61B 1/00128; A61B 1/00034

(57) ABSTRACT

Various embodiments include an endoscopic device. A sheath may enclose the device and may thereby guard against environmental contamination. The sheath may include one or more channels for transmission of instruments and/or solution to and from the body. The sheath may be disposable. A stopper unit may reduce fluid leakage from inside a body cavity during a procedure. The stopper unit may include a serrated outer contour to assist in catching at a body orifice. The stopper unit may have a perforable inner barrier designed to reseal about a probing portion inserted through the stopper unit.

### RELATED APPLICATIONS

The present application claims the benefit of priority of: U.S. provisional patent application No. 62/287,005, entitled “MEASUREMENT DEVICE”, and filed on Jan. 26, 2016; U.S. provisional patent application No. 62/290,440, entitled “MEASUREMENT DEVICE”, and filed on Feb. 2, 2016; and U.S. patent application Ser. No. 15/338,408, entitled “MEASUREMENT DEVICE”, and filed on Oct. 30, 2016, the entirety of each of which is incorporated herein for all purposes.

### BACKGROUND

Various medical procedures are used to examine certain parts of the body, including internal areas. These may be grouped generically into endoscopy procedures. Within the general class of endoscopic procedures, a hysteroscopy examines the uterus, a cystoscopy examines the urinary bladder, a gastroscopy examines the esophagus, stomach, and/or small intestine, a bronchoscopy examines the throat, larynx, trachea, and/or lower airways, a sigmoidoscopy examines the rectum, a colonoscopy examines the rectum and/or colon, a colposcopy examines the cervix, vagina and/or vulva, a nasal endoscopy examines the nasal and sinus passages, etc.

Traditionally endoscopic procedures are performed with expensive equipment. Such equipment may include endoscopes, hysteroscopes, etc. Following each procedure, the equipment may require sterilization. The sterilization must generally be performed in a hospital. Thus, traditionally, endoscopic procedures have been performed in a hospital setting.

When procedures are performed in a hospital, there are a number of associated expenses, including preparing and sterilizing the operating room, scrubbing down for the surgeon and other medical professionals, coats and gowns worn by the participants, etc. There are fees to rent the room, there are fees for the doctor, and there are fees for the anesthesiologist. Additionally, there is extra time required by both the doctor and the patient to travel to the hospital. There may be further time required if there has been an emergency or other incident whereby the operating room has remained occupied, forcing the doctor and patient to potentially wait for hours. All told, endoscopic procedures performed in hospitals may cost thousands of dollars.

Additionally, even when endoscopic equipment is sterilized and used in hospital settings, the sterilization procedure is not always performed properly and/or does not succeed in destroying all infectious agents. Thus, endoscopic procedures have been known to transmit infections from one patient to another, causing additional harm, suffering, expense, and liability.

There have been attempts to make portions of endoscopes disposable, thus avoiding the need for equipment re-sterilization, and thereby allowing the procedure to be performed in a doctor's office rather than in a hospital setting. However, endoscopes incorporate expensive electronics, and disposing of such electronics after every use is still costly.

As endoscopes are often used to image organs and body cavities, a light source is typically required. The light source is traditionally placed at the tip of the endoscope. However, the light source can also generate heat. The light source may end up burning the patient.

### SUMMARY

Various embodiments include a device for performing an endoscopic procedure. Various embodiments include a hysteroscope. Various embodiments include a cystoscope. Various embodiments include a colonoscope. Various embodiments include a gastroscope. Various embodiments include a colposcope.

According to various embodiments, the endoscope includes a probing portion, and a separate sensor assembly. The probing portion may be used for insertion and/or direct contact with the patient. The sensor assembly may include a camera and other electronics for obtaining data from the procedure. The sensor assembly may be shielded from any direct contact with the patient. In various embodiments, the probing portion may be disposable, while the sensor assembly may be reused. Since the sensor assembly never comes into contact with the patient, the highest level sterilization procedures need not be used, and infection risk can be minimized.

According to various embodiments, a device includes a probing portion for direct insertion into a body cavity. The probing portion is brought into proximity to the tissue and/or area that is to be examined. According to various embodiments, the device further includes a sensor assembly, which receives signals from the probing portion and processes, records, interprets, and/or display such signals.

In various embodiments, the probing portion comprises a cannula. The probing portion may be long, thin, and semi-rigid. The probing portion may include a tip. The tip may have a surface that is at an angle to the cross-sectional plane of the probing portion. The probing portion may include one or more optical fibers running parallel to the long axis of the probing portion. In some embodiments, there may be hundreds of such fibers. In various embodiments, the optical fibers may carry light from the tip of the probe to the sensor assembly. In some embodiments, the optical fibers may also carry light from a light source to the probe, whereupon the light may reflect off the surrounding tissue, return to the top, and be carried back via fiber optics to the sensor assembly.

In various embodiments, the probing portion may further include a lens on the tip. The lens may change the focus of incoming light (e.g., of reflected light) so that the light travels down the length of the probing portion to the sensor assembly.

In various embodiments, the probing portion may include a tube for inserting liquid (e.g., saline solution) into the area being examined.

In various embodiments, the probing portion may include a tube for removing liquid (e.g., saline solution) from the area being examined.

In various embodiments, the probing portion may include a tube through which instruments may be inserted to reach the area being examined. Such instruments may include forceps, such as forceps to excise a portion of tissue during a biopsy.

In various embodiments, the probing portion may include a back-flow stopper. The back-flow stopper may encircle the probing portion along some of its length. The back-flow stopper may have a tapered profile with grooves, giving it a gradually expanding diameter as seen from the tip of the probing portion to the opposite end of the probing portion. The back-flow stopper may thus cause the probing portion to wedge in place at a certain point, e.g., when the back-flow stopper catches within the cervix. The back-flow stopper may serve to fill the entire cross section of a particular body area (e.g., a cervix), and to thereby prevent any fluids from escaping.

In various embodiments, the probing portion may include an instrument port. The instrument port may allow insertion of a surgical instrument (e.g., forceps), which may then be snaked through to the tip-area of the probing portion where the instrument may be brought into contact with patient tissue (e.g., for the purposes of a biopsy).

In various embodiments, the probing portion may include a hollow or tube in its shaft that is closed near the tip, but open near the sensor assembly. Accordingly, components can be inserted into tube to reach the inside of the patient, while still being shielded from direct contact with the patient via the external probing portion. Such components may be part of the sensor assembly, or such components may be separate modules. In various embodiments, optical fibers may be inserted into the tube. In various embodiments, a camera or camera module may be inserted into the tube. In various embodiments, a light source may be inserted into the tube. In various embodiments, optical fibers may be reused. In various embodiments, a camera module may be reused. In various embodiments, a camera may be reused. In various embodiments, a light source may be reused.

In various embodiments, a sheath may enclose an endoscopic device. The sheath may keep the endoscopic device sterile during a procedure. The sheath may be disposable.

In various embodiments a sheath may include a cap that covers a tip of the sheath. The cap may include protruding portions designed to fit into lumens of the sheath. The cap may include a transparent region. The cap may include a hole or hollow allowing passage of fluid, equipment, etc.

In various embodiments, a charging dock may provide a resting place where an endoscopic device and/or a backup battery pack may charge.

### BRIEF DESCRIPTION OF THE DRAWINGS

FIGS. 1-25 each provide an illustration of a respective device, according to some embodiments.

FIG. 26 provides an illustration of a block diagram of a device, according to some embodiments.

FIG. 27 provides in illustration of a device with a sheath, according to some embodiments.

FIG. 28 provides in illustration of a device, according to some embodiments.

FIG. 29 provides in illustration of a device and a dock, according to some embodiments.

FIG. 30 provides an illustration of a device component, according to some embodiments.

FIG. 31 provides in illustration of a device with an angled tip, according to some embodiments.

FIG. 32 provides an illustration of a device component, according to some embodiments.

### DETAILED DESCRIPTION

As used herein, titles, section headings and the like are included for the purposes of clarity, but are not intended to be limiting in any way. For instance, embodiments described in a particular section are not to be assumed to be limited by the title or heading of that section. Further, embodiments described within a particular section (e.g., in the “Summary” section) are not intended to be limiting with respect to contemplated embodiments. It will be understood that embodiments may be contemplated, even embodiments related to a particular section, even if they were not explicitly described in that section.

As used herein, references to devices, servers, storage devices, etc., may include single physical devices or may include devices with multiple separate physical components, or to devices which are instantiated virtually. For example, a server may refer to a single physical device, to a distributed system comprising multiple separated physical components, or to a virtual server residing in software and not tied to any particular hardware device or system. A server may include a cloud server, for example.

As described herein, communication between devices may occur via any suitable means, including via landline, wired, or wireless communication, via the Internet, via telephonic networks, via cellular networks, via satellite communication, via cable, via fiber optic, via local area network, via wide-area network, or via any other means.

### Device

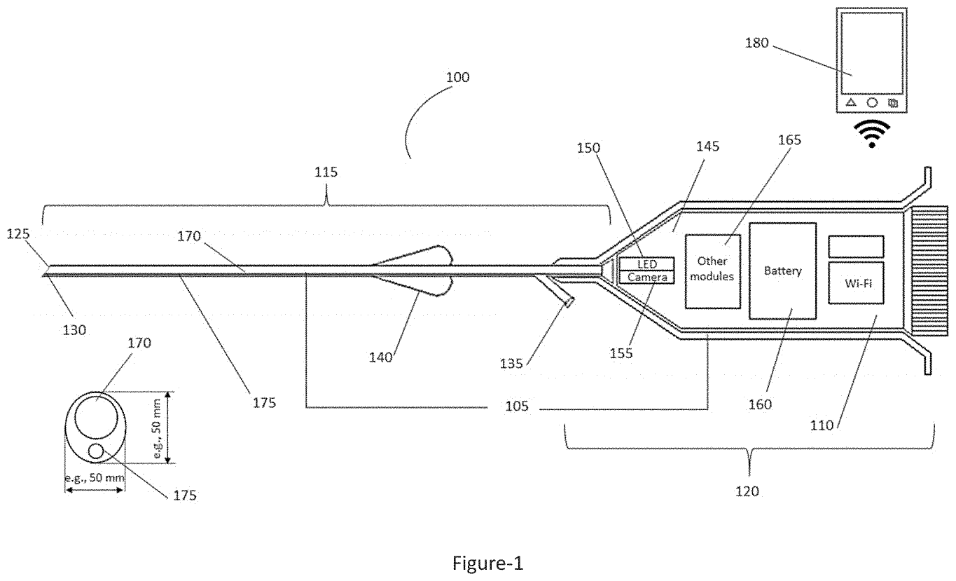

With reference to FIG. 1, a device 100 is shown according to some embodiments. The device includes a probing portion 105 and a sensor assembly 110. The probing portion 105 may include a shaft 115. The shaft may include a cannula. The shaft may include one or more tubes or tunnels running the length of the shaft. The shaft may include tubes such as those made by Welset, A.P. Extrusion, Microlumen, Precision Extrusion, Inc, Raumedic, and Zeus Polymer Extrusions. The shaft may include multi lumen tubing.

The shaft may include one or more optical fibers running the length of the shaft. The shaft may be connected to a housing portion 120, which may be shaped in such a way as to surround, hold and/or incorporate the sensor assembly. The housing portion may resemble a cone in that it may open from a small point of contact with the shaft into a larger, hollowed area into which sensor assembly may be inserted. As will be appreciated, the housing portion may take any suitable shape or geometry in various embodiments.

The probing portion may include a tip 125. The tip may be the leading part of the probing portion as it is inserted into a patient, and may be the point of closest distance or contact with tissue of interest. A lens 130 may be attached to the tip. The lens may focus and/or alter the course of incident light, and direct such light down the length of the shaft until it can reach the sensor assembly at the other end.

The probing portion may include a port 135. The port may be a hollow tube or tube section that branches from the shaft 115. The port may interface to a hollow tunnel or tube within the shaft. The tube may run the length of the shaft all the way from the port 135 to the tip 125. In various embodiments, the port may allow for injection of fluid into the patient, such as injection of saline solution. The fluid may be used to prop open the walls of an organ (e.g., of the uterus) that would normally be collapsed.

The probing portion may include a backflow stopper 140. The back-flow stopper may encircle the probing portion along some of its length. The back-flow stopper may have a tapered profile with grooves, giving it a gradually expanding diameter as seen from the tip of the probing portion to the opposite end of the probing portion. The back-flow stopper may thus cause the probing portion to wedge in place at a certain point, e.g., when the back-flow stopper catches within the cervix. The back-flow stopper may serve to fill the entire cross section of a particular body area (e.g., a cervix), and to thereby prevent any fluids from escaping.

In various embodiments, the backflow stopper 140 can be moved (e.g., slid) along the length of the shaft 115. The backflow stopper can thereby allow the tip to reach a predetermined distance beyond a location where the backflow stopper is expected to catch (e.g., at the cervix). The backflow stopper may make water-tight contact with the shaft 115, so that it may be movable but still not permit fluids from leaking between it and the shaft.

The probing portion 105 may be made from one or more materials. Such materials may include PVC (Polyvinyl Chloride), PES (Polyether sulfone), PTFE (Polytetrafluoroethylene), PE (Polyethylene), PEEK (Polyetheretherketone), PS (Polysulfone), PC (Polycarbonate), and/or FEP (Fluorinated Ethylene Propylene).

### Sensor Assembly

In various embodiments, the sensor assembly 110 includes a chassis 145. The chassis may be any suitable structure which may hold electronics, sensors, and/or other components. The chassis 145 may be made of metal, plastic, and/or any other suitable material. The chassis 145 may be hollow, and may have built-in holes, screw threads, clamps, fasteners, brackets, or other means for attaching electronics, etc. The outer surface of the chassis may be shaped in a way that is complementary to the housing 120 of the probing portion 105. In this way, the sensor assembly 110 may fit into the housing 120 of the probing portion 105. The fit may be a snug fit. In various embodiments, a locking mechanism secures the sensor assembly once inserted into the housing 120.

In various embodiments, sensor assembly includes a light source 150. The light source may be a light-emitting diode (LED), an organic LED (OLED), a quantum dot, or any other suitable light source. In various embodiments, the light source 150 may be Dual Tone Flash LED Lighting. The light source may be located proximately to the back end of the shaft 115. The light source may be oriented so as to direct light down the length of the shaft. The light may thereby emerge from the tip 125 and illuminate surrounding tissue.

In various embodiments, sensor assembly includes a camera 155. As will be appreciated, camera 155 may be any image sensor and/or light sensor. Exemplary sensors include the Omnivision-OV6946 (Omnivision-OV 6930) the Cmosis Naneye 2D, and the Micro Scoutcam LED 3.45, and/or any other suitable sensor. Camera 155 may comprise a charge coupled device (CCD) sensor, a complementary metal oxide semiconductor (CMOS) sensor, and/or any other suitable sensor. Camera 155 may further include any suitable optical or lens assembly and/or any other appropriate components.

Camera 155 may be located proximate to the back end of the shaft 115. The shaft may carry light from the tip of the shaft, down along its length, to the back end of the shaft, where such light may then be received by the camera 155. The camera may thereby capture images from the tip of the shaft 155, even though the camera itself may be remote from the tissue being imaged.

In various embodiments, sensor assembly includes a power source 160. The power source may be a battery, ultra-capacitor, fuel cell, or any other suitable power source. The power source may be a re-chargeable battery. The power source may be a power supply unit, which may in turn be plugged into the electrical grid. As will be appreciated, various embodiments contemplate that power may be supplied via any suitable mechanism.

In various embodiments, device 100 may include a charging cradle. The sensor assembly may be placed into the charging cradle, at which point the battery in the sensor assembly may interface to the cradle and recharge.

### Other Modules

In various embodiments, sensor assembly 110 includes one or more additional components or modules. These are denoted by reference numeral 165.

In various embodiments, sensor assembly 110 may serve as an embedded and/or special purpose computer. Accordingly, sensor assembly may execute one or more algorithmic and/or data-processing steps in accordance with various embodiments. Algorithms may include receiving incident light into the camera 155, processing the incident light, generating images (and/or videos), storing the images, and/or transmitting images to an external device.

In various embodiments, a storage device may store computer instructions, program instructions, and/or program data. A storage device may be a solid state drive (SSD), hard disk, flash drive, or any other suitable storage device. In various embodiments, memory may store data, intermediate calculation values, and/or any other information. Memory may include Random Access Memory (RAM), and/or dynamic random access memory (DRAM), for example.

In various embodiments, a processor may include any circuitry, logic, or the like for executing computer instructions according to various embodiments. A processor may include an integrated circuit, a digital signal processor, a controller, a micro-controller, and/or any other suitable electronics or processing device. Processor may include an Intel x86-type processor, an Advanced Micro Devices (AMD) processor, an ARM processor, a Qualcomm Snapdragon processor, a Digital Signal Processor, and/or any other suitable processor.

In various embodiments, a graphics processing unit (GPU) may perform specialized processing tasks. Such processing tasks may include generating, compressing, and/or encoding images based on incident light received via the shaft 115. GPU may be a processor from NVidia (e.g., the GeForce GTX 960), an AMD Radeon processor, and/or any other suitable processor.

In various embodiments, a communication port allows the sensor assembly 110 to communicate with one or more external devices 180. The communication port may include an antenna. The communication port may include a transmitter, a receiver, and/or a transceiver. The communications port may include a Wi-Fi transceiver, a cellular transceiver, a Bluetooth transceiver, and/or a transceiver according to any other suitable protocol. A communications port may include an Ethernet port, a serial port, and/or any other suitable port.

In various embodiments, a power port may be used for plugging into an electrical grid (e.g., either directly or through an external adapter), into a generator, and/or into any external power source.

In various embodiments, the sensor assembly may include one or more indicator lights. For example, an indicator light might come on if power is running low, if a component is malfunctioning, if the sensor assembly is not secure in housing 120, or upon any other occurrence.

In various embodiments, the sensor assembly may include a speaker or other audio output device. The speaker may output tones, synthetic voice, or other audio indicative of device status or of any other situation or occurrence.

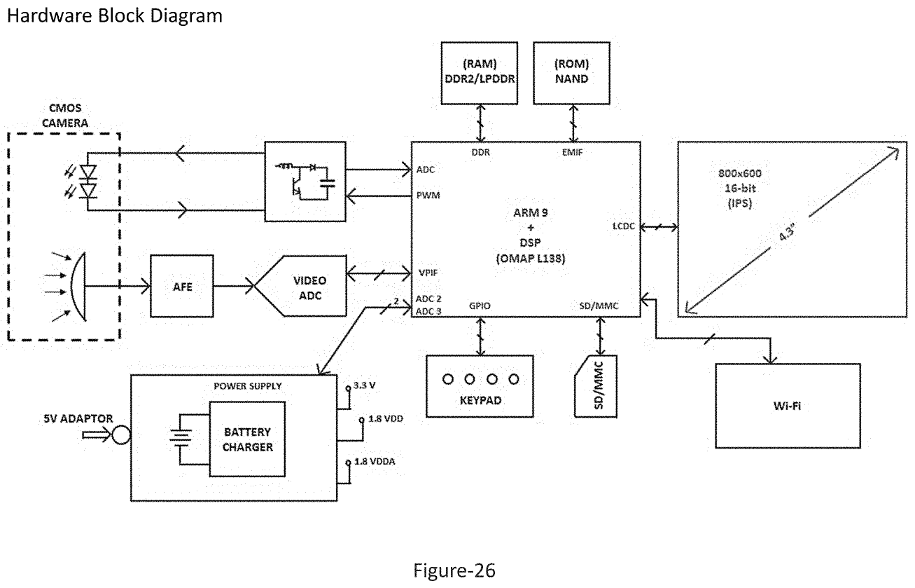

FIG. 26 depicts various device components, according to some embodiments.

Various embodiments are illustrated, depicted, and/or described herein with respect to certain configurations and/or arrangements or parts. However, it will be appreciated that contemplated embodiments are not limited only to the arrangements described, but include any suitable arrangements. In various embodiments, certain components may be combined. In various embodiments, certain components may be separated. In various embodiments, a single component may be replaced by multiple components. In various embodiments, multiple components may be replaced by a single component. In various embodiments, additional components may be used. In various embodiments, some components may be omitted. In various embodiments, the order, position, or arrangement of components may be altered. Various embodiments contemplate any suitable arrangement, configuration, ordering, and/or combination of components that can carry out the functionality of one or more embodiments.

### Illustrative Hardware Specification

The following is an illustrated hardware specification for a device, according to various embodiments.

### Camera and LED Lighting:

CMOS Camera:—OMNI VISION 400×400 resolution, 30 fps
AFE:—Video Comb Filter, Video Amplifier, Voltage Clamber
Video ADC:—PAL/SECAM 8-bit Video Decoder
LED Driver:—Dual tone dimmable LED FLASH

### CPU and DSP Module:

Processor:—Texas Instruments OMAP L138(ARM9+C6000)
RAM:—128 MB DDR2
ROM:—4 Gb NAND FLASH ROM
Storage:—Micro SD Card (up to 32 GB)
Android ready (optional)
4-5 Keypads
800×600, 16 bit IPS Capacitive Touch Display
Wi-Fi Streaming
USB 2.0
PB free RoHS Components—Safety standard
6 layer PCB—Reduced EMI emission

### Battery and Charging

Rechargeable Li/ion or Li/polymer 3.7V, 2500 mAh Battery
Dock charging

### Optical Fibers

Various embodiments include one or more optical fibers 170. The optical fibers may include plastic fibers, glass fibers, clear fibers, or any other suitable fibers. The fibers may be capable of transmitting light. The fibers may be capable of transmitting light with minimal losses. The fibers may be capable of transmitting light even when bent or curved.

The optical fibers 170 may run the length of the shaft 115. In various embodiments, the optical fibers carry light from the light source 150 to the tip 125. Light may escape from the tip and illuminate surrounding tissue.

In various embodiments, the optical fibers carry light from the tip 125 to the back end of the shaft. There, the light may reach the camera 155, which may thereby capture images of the tissue from near the tip 125.

In various embodiments, by transmitting light over a distance, the optical fibers 170 allow the light source 150 to remain remote from the tissue being imaged. This eliminates the chances that a patient will be burned by heat from the light source.

In various embodiments, by transmitting light over a distance, the optical fibers 170 allow the camera 155 to remain remote from the tissue being imaged. The camera, which may be comparatively expensive, may thus be kept isolated in a sterile environment (e.g., within the sensor assembly 110), and may thereby be readily re-usable. This can result in significant benefits in terms of cost reductions for endoscopic procedures.

A device according to various embodiments may employ multiple optical fibers 170. The quantity of such fibers may number in the hundreds or more. The optical fibers may be situated parallel to one another along the length of shaft 115, and may avoid any twists, weaves, etc. In this way, incident points of light from the tip do not get scrambled once they reach the back end of the shaft 115.

In various embodiments, a tube or tunnel may house the optical fibers. The diameter of this tube may be 1.65 mm, in some embodiments. In some embodiments, other diameters may be used.

### Lens

In various embodiments, a lens is attached to the tip 125. The lens may be plastic, glass, or any suitable material. The lens may be made of refractive material. In some embodiments, a diffraction grating or other suitable optical mechanism may be employed.

The lens may alter the course of incoming light so that converging light rays entering the lens exit the lens as parallel light rays. These parallel light rays may be directed into the optical fibers, and may thus traverse the length of the fibers to reach the camera 155. A lens or other optics proximate to, or associated with the camera may once again cause the parallel light rays to converge (i.e., focus the light rays) so that a proper image may be formed.

The lens may be glued to the ends of the optical fibers 170, or attached via any suitable mechanism. The lens may simultaneously interface to multiple (e.g., to all) of the optical fibers 170. In various embodiments, one or more optical fibers themselves may be shaped at their ends to form a lens.

In various embodiments, a fisheye lens may be employed. The fisheye lens may provide a wider field of view than does a standard lens.

In various embodiments, other types of lenses may be employed, depending on the desired application, field of view, or any other criteria.

### Tip

In various embodiments, the tip 125 has flat or substantially flat surface which is not strictly perpendicular to the axis of the shaft. Rather, the surface may be at an angle to this perpendicular plane. The angle may be 30 degrees, in some embodiments. In some embodiments, the angle may be some other number of degrees. The angled tip may provide a larger surface area with which to take in light and may allow for imaging of a larger area of tissue. The angled tip may also allow imaging of areas that are above, below, and to the side so the shaft 115, rather than strictly in front of it (i.e., in the line of its axis). Further, by rotating the device about its axis, the operator (e.g., the doctor) may sweep the surface of the tip, thereby allowing viewing of a 360 swath surrounding the axis of the shaft.

### Interface Between Probing Portion and Sensor Assembly

In various embodiments, an optic fiber coupler is disposed at the back end of the optical fibers, and is designed so as to be adjacent to the light source and/or camera on the sensor assembly 110. The optic fiber coupler may have optical properties. For example, it may be a lens or prism. It may serve to widen or narrow the beam(s) of light coming from the optical fibers and entering the camera. It may focus the beam(s) of light into the camera. It may filter the light entering the camera. For example, it may narrow the beam of light coming from the optical fibers if the collective diameter of the optical fibers is greater than the aperture of the camera.

The optic fiber coupler may also widen, narrow, filter, or otherwise modify the light emitted from the light source prior to entering the optical fibers.

### Backflow Stopper

In various embodiments, the backflow stopper 140 may perform one or more functions. The backflow stopper may block an area or opening completely. It may thus prevent any fluid from leaking out. In various embodiments, saline solution may be injected into an area, such as to prop open the walls of the surrounding organ. It may be desirable that the saline solution does not leak out, so that the area may remain open, so that solution is not wasted, so that time is not wasted replacing lost solution, and/or so that any mess or waste product is minimized.

In various embodiments, the backflow stopper 140, once wedged into a particular location in the body, may hold the entire device 100 in place. It may thus prevent the tip 125 from going further than desired.

In various embodiments, a backflow stopper may have the rough shape of a cone or conical wedge. The backflow stopper may have grooves. With this configuration, the backflow stopper may be inserted progressively deeper into a particular opening in the body, until the diameter of the backflow stopper just matches the size of the opening. The backflow stopper may thus fill the opening, seal the area from fluid leakage, and/or fix the device 100 in place.

In various embodiments, the backflow stopper 140 has a step-like, serrated, grooved, or terraced surface. Each “step” may form a ring of a particular diameter around the axis of the shaft 115. Thus, the diameter of the backflow stopper may not increase strictly linearly long its axis, but may rather increase in a step-like function. This may create a ratchet effect whereby the backflow stopper is not as easily withdrawn from an area as it was inserted. This may help to fix the backflow stopper in place, and/or maintain a water-tight seal to avoid fluid leakage.

Although the backflow stopper has been depicted with respect to some representative shapes and geometries, it will be appreciated that various embodiments contemplate any suitable shape and/or geometry for the backflow stopper. For example, the backflows topper need not increase in diameter as a linear function of distance along its axis, but may rather increase in an arc-like function. Various embodiments contemplate any suitable shape that would allow the backflow stopper to perform one or more functions described herein.

In various embodiments, a backflow stopper may fit over a sheath that covers a probing portion, rather than fitting directly over a probing portion. In various embodiments, the backflow stopper may be attached to the sheath. In various embodiments, the backflow stopper may be slidable along all or a portion of the length of the sheath (e.g., along the portion that covers the probing arm). In various embodiments, the backflow stopper may have an internal membrane or barrier that blocks the channel running through its center. This barrier may be punctured/penetrated by the sheath when the sheath (and e.g., the internal probing portion) is inserted through the backflow stopper.

Various embodiments include a backflow stopper with a barrier. The backflow stopper may comprise a structural portion that is substantially cylindrically symmetrical about a central axis, the structural portion having a cross sectional profile that substantially increases in outer diameter over at least a portion of a length of the central axis. The backflow stopper may thereby have a gradually expanding width or circumference so that it can act as a wedge or stopper in order to be able to plug orifices of varying width.

The backflow stopper may include a substantially hollow channel running along the central axis through the structural portion, the hollow channel permitting the admittance of a probing portion of an endoscopic device. The backflow stopper may further include a barrier closing off at least a portion of the hollow channel, the barrier comprising a material that is penetrable by the probing portion of the endoscopic device.

In various embodiments, the barrier comprises silicone gel. In various embodiments, the barrier comprises a silicone membrane. In various embodiments, the barrier comprises a membrane. However, it will be appreciated that any suitable barrier may be used.

In various embodiments, the barrier is operable to seal around the outside of the probing portion after penetration by the probing portion. The barrier may yield enough to allow the probing portion (or its surrounding sheath) to penetrate through the barrier. However, the barrier may thereafter expand or conform to the sheath so as to create a watertight or substantially watertight barrier around the sheath.

In various embodiments, the backflow stopper may include a structural portion that is substantially cylindrically symmetrical about a central axis, the structural portion having a grooved outer surface with concentric grooves forming circles about the central axis.

The concentric grooves may successively increase in diameter over at least a portion of a length of the central axis. As will be appreciated, the local shape of the successive grooves may cause the overall diameter to fluctuate from “peaks” to “troughs” at a local level. However, when viewed from the vantage point of successive “peaks” or successive “troughs”, the grooves may be of increasing diameter over at least a portion of the length of the central axis.

In various embodiments, the grooves are concentric and non-intersecting. That is, there is no connection among successive troughs of the grooves. Also, in various embodiments, there is no spiral, helix, or other similar pattern whereby a single groove wraps continuously in spiral-like fashion. Such a spiral setup might allow fluid to escape by spiraling through the trough of the single groove. Rather, in various embodiments, the grooves are concentric but do not meet or intersect.

### Ports

Various embodiments include a port 135. The port may comprise a branch from the shaft 115, where the branch includes a hollow tube with an opening. The hollow tube may connect with saline path 175, which may be a hollow tube or tunnel within the shaft 115. Thus, the port may be used as an access point to the shaft 115. The saline path 175 within the shaft may lead all the way to the tip 125, where it may open into the ambient surroundings.

In various embodiments, port 135 is used to inject fluid (e.g., saline solution) into the shaft. The saline fluid travels the length of the shaft to the tip 125, and is thereby injected into the patient.

The port 135 may be angled with respect to the shaft 115. The port 135 may form an obtuse angle with respect to the shaft. The port may thereby minimize any change in direction experienced by fluid or other matter inserted into the port 135 on its way to the tip 125. As will be appreciated, the port may be connected to the shaft via some other geometry, such as via an arcing section of tube.

In various embodiments, the saline path may have a diameter of 0.5 mm. However, various embodiments contemplate other diameters.

Various embodiments include additional and/or alternative ports. Such ports may interface to separate tubes or tunnels within the shaft.

In various embodiments, a suction port 310 is used to withdraw fluid (e.g., saline solution) from the shaft. The suction port 310 may connect to another tube or tunnel within the shaft which may run all the way to another opening in the tip 125. Fluid from inside the patient may enter the opening to such tunnel or tube at the tip 125, travel down the length of the shaft 115 to the port, and then come out the opening in the suction port 310.

In some applications, saline solution is injected into a patient, and then withdrawn through suction port 310. The amount of saline solution withdrawn may be measured and compared to the amount of saline solution that was injected. If the amount of saline solution withdrawn differs from the amount injected, then there may be a problem indicated.

Various embodiments include an instrument port 210. The instrument port may likewise connect to a hollow tube or tunnel within the shaft, which then opens at the tip of the shaft. In some applications, an instrument may be inserted into the instrument port, and threaded through the length of the shaft to emerge at the tip. One or more operations may then be performed with the instrument, such as taking a tissue biopsy. The instrument may then be withdrawn through the same instrument port, potentially including any tissue sample obtained.

Various embodiments may include one or more ports. Various embodiments may include one or more of the aforementioned ports. Various embodiments may include any combination or sub-combination of the aforementioned ports. For example, some embodiments may include a shaft with just a saline port, some embodiments may include a shaft with both a saline port and a suction port, some embodiments may include only an instrument port, some embodiments may include both a saline port and an instrument port, and some embodiments may include saline, suction, and instrument ports.

Various embodiments contemplate additional quantities and/or types of ports that may be used separately and/or in addition to the aforementioned ports.

In various embodiments, a single port may permit multiple functions. In various embodiments, a single tunnel, tube, or path may permit multiple functions. For example, a single port may permit the injection of a saline solution and the insertion of an instrument through the same tube or tunnel.

In various embodiments, suction port 310 may have a diameter of 0.5 mm. In various embodiments, instrument port 210 may have a diameter of 2 mm. In various embodiments, a port used for both suction and instruments may have a diameter of 2 mm. In various embodiments, other diameters may be used for the aforementioned ports.

### Double Packaging Procedure

In various embodiments, device 100 is packaged in two-stage packaging. Opening a first stage of the packaging will expose only the housing 120 portion of device 100. Once the housing portion is open, sensor assembly 110 can be inserted into the housing 120. Once the sensor assembly has been inserted, the doctor can wash his hands, switch gloves, or otherwise decontaminate his own hands. The second stage of the packaging can then be opened, which will expose the shaft 115 and tip. The portion of the device that will enter the patient thereby is exposed to minimal contamination risk from the sensor assembly 110.

### Separate Device, Screen, Etc.

In various embodiments, an external device 180 receives communications from device 100. The external device may be a computer, laptop, cellular phone, tablet, iPad, iPhone, Android device, personal computer, dedicated computer, smart phone, or any other device. The external device 180 may receive communications via Wi-Fi, Bluetooth, or via any other means. Communications may include image data (e.g., image data captured from the patient), video data (e.g., video data captured from the patient), streaming data (e.g., streaming video) or any other data.

The external device 180 may include a display screen. The external device may display images and/or video received from device 100. In various embodiments, the external device 180 may display such data in real time. In various embodiments, the doctor performing the procedure may view the display screen in order to see the internal view from tip 125 of the device 100. The doctor may thereby take action, including moving the device 100, taking a biopsy, injecting saline solution, withdrawing saline solution, and/or any other action.

According to some embodiments, the device 100 may include Real-time Video streaming to tablet/iPad/PC/Cloud over Dual Band Wi-Fi (2.4 GHz and 5 GHz).

In various embodiments, where the display screen is separated from the device 100, there is provided an operational advantage in that the device 100 can be rotated about its long axis without also rotating the display. The doctor thereby doesn't have to worry about craning or straining his neck in order to see the images or videos in the upright and/or desired orientation.

In various embodiments, the external device 180 may store or direct the storage of data received from device 100. The external device may store data on a local drive or server. The external device 180 may also transmit data for storage in a remote or cloud server. As will be appreciated, various embodiments contemplate that obtained data may be stored in any suitable location and in any suitable fashion.

### Advantages

Devices according to various embodiments may offer various advantages. With electronics and other costly components kept out of contact with the patient, there is no need to dispose of these after a single use. Instead, only probing portion 105 can be disposed of after each use. The probing portion may comprise only relatively cheap components, such as plastics.

Where electronics and other components are maintained at a distance, the shaft 115 and tip 125 may be kept narrow. This may allow for greater ease of entry into the patient, greater flexibility, and/or greater comfort for the patient.

With electronics maintained at a distance, particularly the light source, heating at the tip is substantially eliminated. The potential for burns to a patient may thereby be greatly reduced. This may further allow procedures to last longer, since there is little risk of heat build-up. This may be especially important in a teaching setting, where a procedure may last for an extended duration as students are able to watch and as different steps are explained.

With electronics maintained at a distance, and as part of a separate assembly (e.g., as part of the sensor assembly 110), there is the potential to swap out electronics during the middle of a procedure without having to withdraw the device 100 from the patient. For example, during a procedure, the power source may run out, the light source 150 may burn out, the camera 155 may cease to function, and/or any other disruption may occur. At this point, the sensor assembly 110 may be removed from the housing 120 even while the shaft 115 and tip 125 remain inside the patient. A new sensor assembly may then be inserted into housing 120. The ability to swap out the sensor assembly 110 may save time and result in greater comfort for the patient. It may also assure that the tip is not moved from a desired location, which would otherwise have to be located again.

Since the probing portion (including, e.g., a cannula) is sterile, making it single use eliminates the need for costly sterilization and minimizes risk of infection.

### Trade Names

A device according to various embodiments that is used for performing hysteroscopy may be branded, marketed as, or otherwise named “Hysto-view”.

A device according to various embodiments that is used for performing cystoscopy may be branded, marketed as, or otherwise named “Cysto-view”.

A device according to various embodiments that is used for performing gastroscopy may be branded, marketed as, or otherwise named “Gastro-view”.

A device according to various embodiments that is used for performing colonoscopy may be branded, marketed as, or otherwise named “Colono-view”.

A device according to various embodiments that is used for performing colposcopy may be branded, marketed as, or otherwise named “Colpo-view”.

A device according to various embodiments that is used for performing endoscopy may be branded, marketed as, or otherwise named “EZ-view”.

A device according to various embodiments that is used for performing endoscopy may be branded, marketed as, or otherwise named “Hystosure”.

### Device Control

In various embodiments, the remote device 180 may be used to control one or more aspects of device 100. For example, an operator of the remote device may input commands using a keypad, touch screen, etc. Such commands may include commands to begin recording video, commands to increase or decrease a video resolution, commands to change an aspect of the light being emitted from the light source (e.g., commands to change the intensity or color composition), or any other commands.

Various embodiments may allow for real-time device control by a remote device 180 (e.g., an iPad) over BLE (Bluetooth Low Energy). Various embodiments contemplate that other communications technologies or protocols may be used.

### Some Embodiments

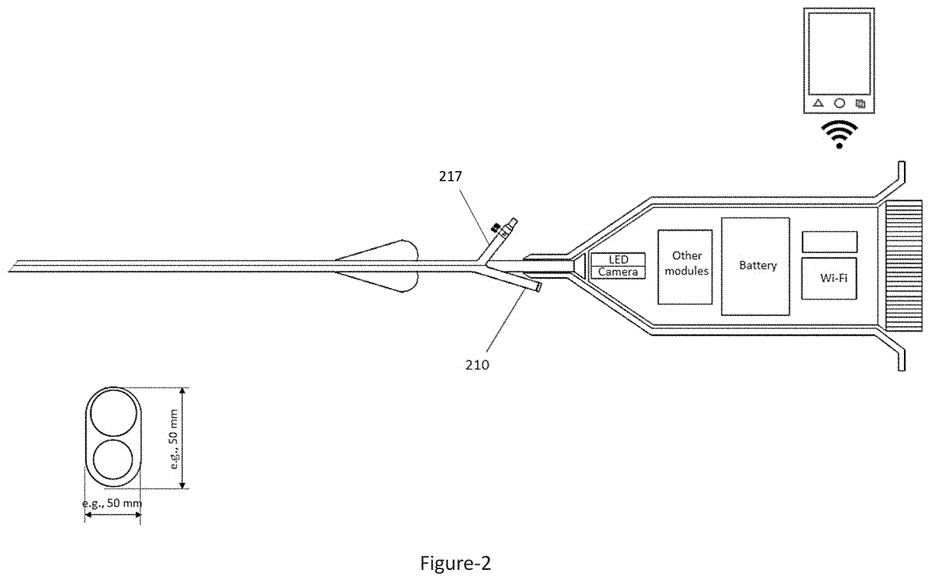

With respect to FIG. 2, a device is shown according to some embodiments. The device includes a port 210 for injection of saline, liquid, or other material or objects. The device includes a port 217 for withdrawal of saline, solution, or other material or objects.

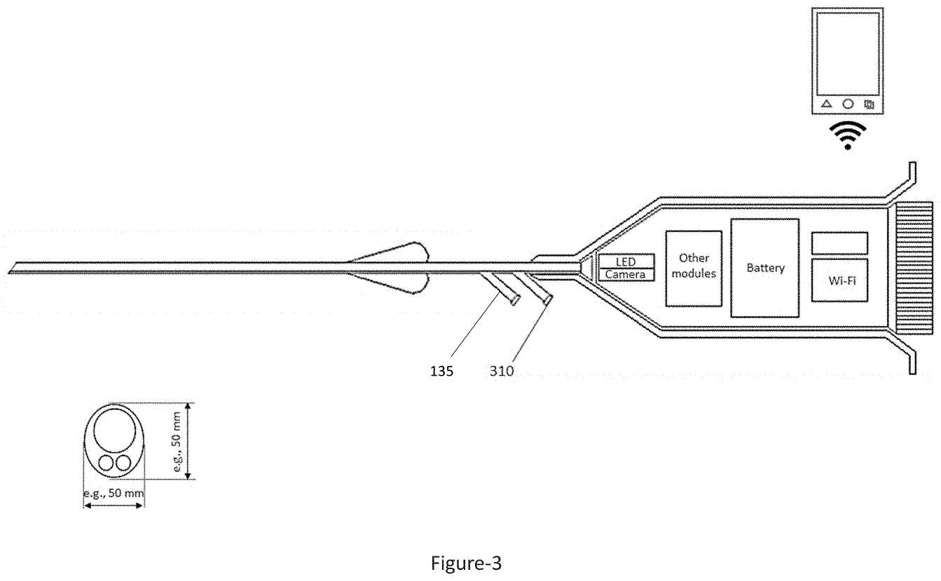

With respect to FIG. 3, a device is shown according to some embodiments. The device includes a second port 310 for injection of saline, liquid, or other material or objects. This port may be in addition to the aforementioned port 135.

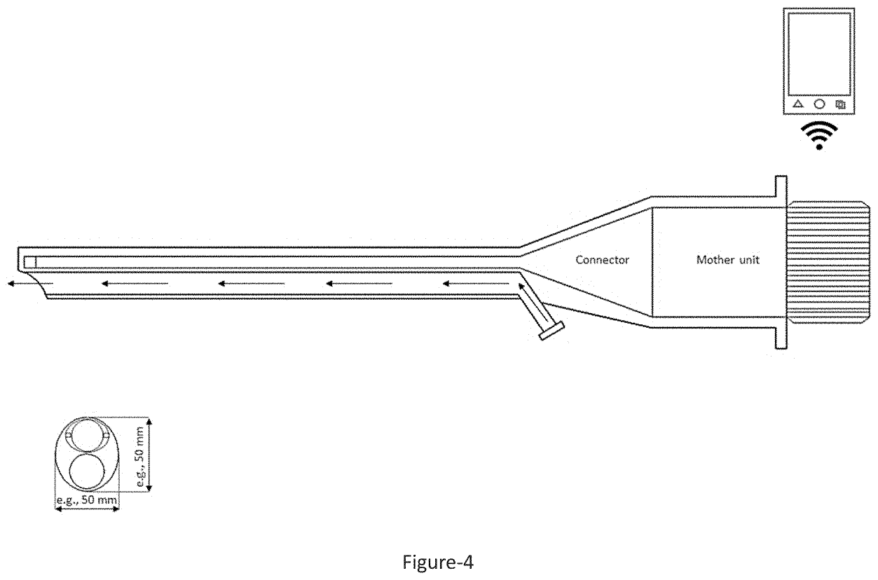

With reference to FIG. 4, a device is shown according to some embodiments. The design comes with removable sheath which can be disposable after single use. Camera and other modules can be reused; hence the overall treatment cost can be reduced. The data can be streamed in to the remote device (e.g., tablet) provided with the product by means of Wi-Fi, or via any other means.

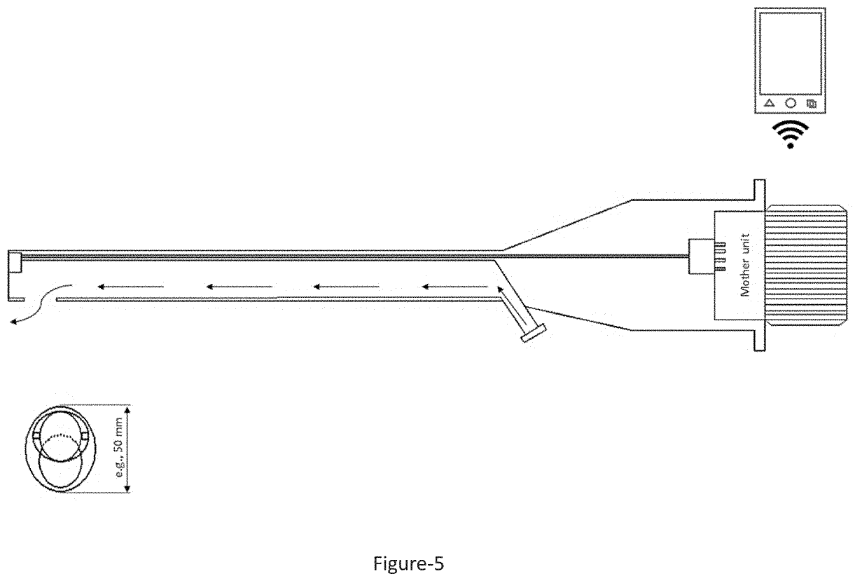

With reference to FIG. 5, a device is shown according to some embodiments. The design comes with disposable camera and LED module. Both saline and instrument ports are integrated in a single tube. The data can be streamed in to the remote device (e.g., tablet) provided with the product by means of Wi-Fi.

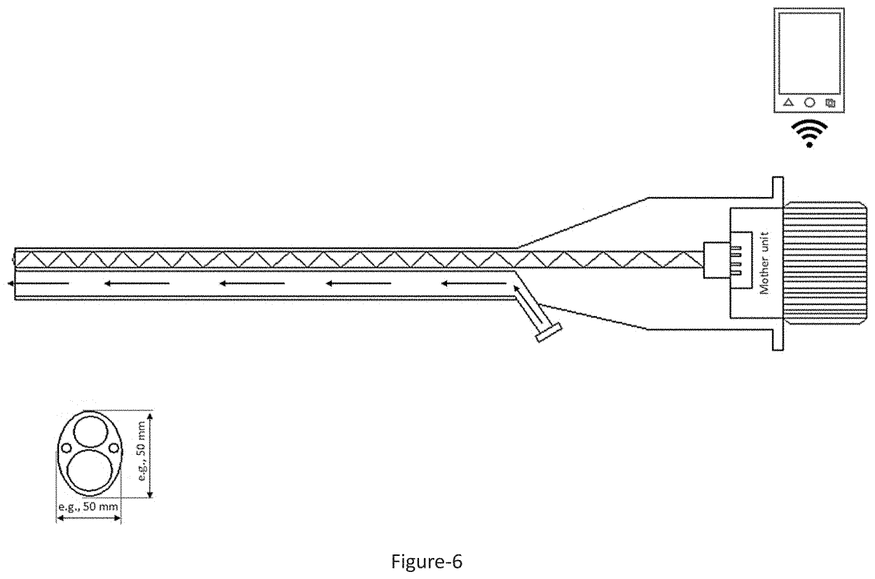

With reference to FIG. 6, a device is shown according to some embodiments. The design comes with disposable fiber optics. Camera and LED module are kept in the mother unit and fiber optics connected to the camera. The lens which is kept on the tip of the device allows the capture of video. So, the camera and other modules can be reused, hence the overall treatment cost can be reduced. The data can be streamed in to the remote device (e.g., tablet) provided with the product by means of Wi-Fi.

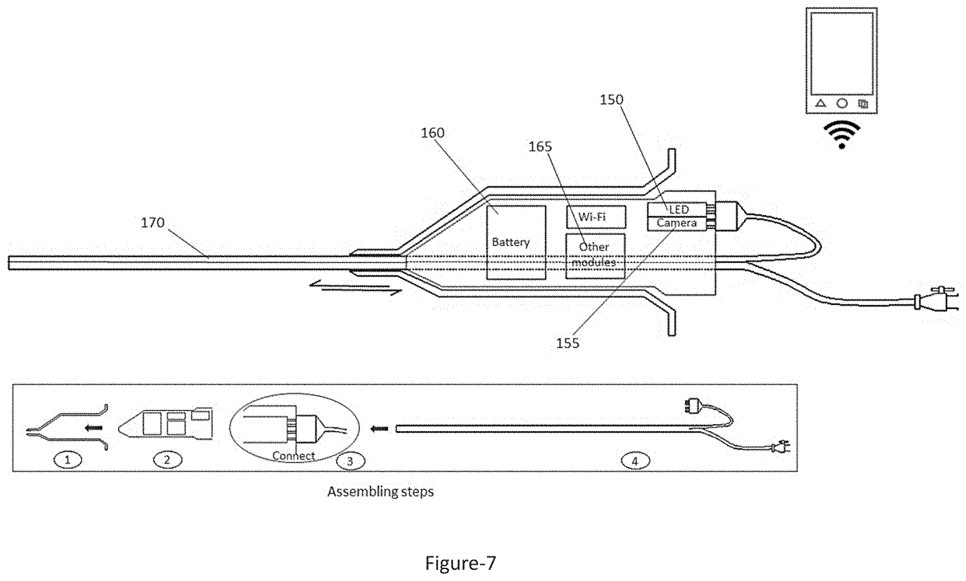

With reference to FIG. 7, a device is shown according to some embodiments. The design comes with disposable fiber optics. Camera and LED module are kept in the mother unit and fiber optics connected to the camera. The lens which is kept on the tip of device allows the capture of video. So, the camera and other modules can be reused, hence the overall treatment cost can be reduced. The data can be streamed in to the remote device (e.g., tablet) provided with the product by means of Wi-Fi.

In various embodiments, the coupler attaching the sensor assembly 110 with the shaft 115 is in the front tip of the sensor assembly 110 (i.e., proximate to the back end of the shaft 115), eliminating the passing of the sterile optic fiber connector through the sensor assembly 110. This keeps the probing portion 105 sterile and not in contact within the sensor assembly 110. This also eliminates making an opening inside the sensor assembly 110, which keeps it sterile and provides for ease of manufacturing, in various embodiments.

In various embodiments, the shaft 115 includes a hollow channel, tunnel or tube. This tube may be open at one end of the shaft, opening into the cavity formed by the housing portion 120. At the other end of the shaft, at tip 125, the tube may be closed off. This may allow the insertion of equipment, components, electronics, etc. into the tunnel in order to be near the tip, but not exposed to the surrounding environment (e.g., not exposed to the patient). In various embodiments, a part of the sensor assembly 110 may be shaped in such a way that it can be inserted into the tube. For example, part of the sensor assembly may be shaped as a long, narrow rod that is slightly less than the diameter of the tube. In various embodiments, equipment, components, electronics, etc. may be separate from the sensor assembly 110. In such cases, the equipment may be designed to interface with the sensor assembly 110, such as by a plug, connector, latch, etc.

Where device 100 incorporates a tube that is never exposed to the patient, there is permitted the insertion of equipment (or portions of equipment) that need not be subjected to the most rigorous sterilization procedures, and therefore need not be disposed of. Accordingly, equipment can be inserted into the patient, yet can also be reused. In some embodiments, the probing portion may thereby effectively serve as a protective covering for equipment being inserted into the patient.

Various embodiments contemplate that relatively expensive equipment, devices, components, etc., may be inserted into the patient. Such components may include a camera and/or a light source. Where a camera is inserted into the patient, light from the patient need not travel far (e.g., the length of shaft 115) before reaching the camera. Where a light source is inserted into the patient, light need not travel far to reach the patient. In various embodiments, such components may then be reused, while the probing portion may be discarded.

In various embodiments, if a camera and/or light source is able to be inserted into the shaft 115 and thereby situated near the tip 125, then optical fibers may not be present. Image and/or video data from the camera may be transmitted down the length of the shaft 115 to the sensor assembly 110 via wires, copper wires, or via any other suitable means. In various embodiments, the camera and/or light source may also be supplied with power from the power source 160 via wires, copper wires, or via any other suitable means.

Insertion of Optical Fibers into Probing Portion

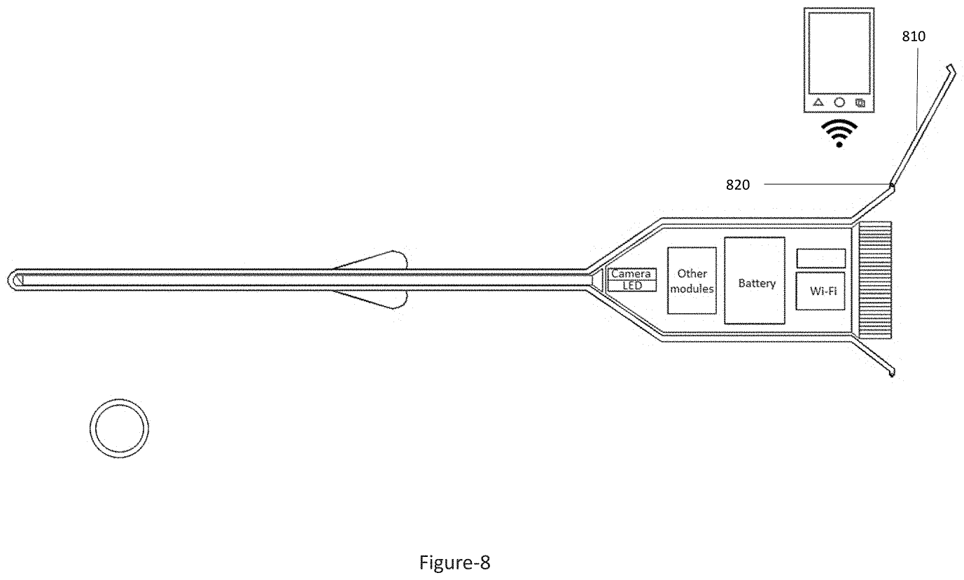

FIGS. 8-16 include various embodiments in which optical fibers are inserted into the shaft 115 of probing portion 105. The optical fibers may avoid direct contact with the patient and may thereby be reused. As will be appreciated, optical fibers may be inserted with support structures, ties, etc. As such, in various embodiments, optical fibers may form a cable assembly.

In various embodiments, electronic components may remain in the main part of sensor assembly 110. Electronic components may remain inside housing portion 120. In various embodiments, the camera may feature the Omnivision-OV 6930.

With reference to FIG. 8, a device is shown according to some embodiments. Various embodiments include a disposable sheath (e.g., probing portion 105) and reusable fiber optics. In such embodiments, the fiber optics may be inserted into the shaft of the probing portion in order to carry light to and from the tip. In various embodiments, the device may be used for imaging the uterus. In various embodiments, the device may be used for other body parts. In various embodiments, the camera and light source may remain in the main part of the sensor assembly 110 and may optically interface with the optical fibers that are inserted into the shaft 115. In various embodiments, the reusable fiber optics include a lens at their terminal ends at the tip. The lens also may be inserted into the shaft along with fiber optics, and may never make contact with the patient due to the surrounding sheath (probing portion 105).

Various embodiments include a cap 810 (not to be confused with the distinct cap 3200). The cap 810 may cover and/or enclose the housing portion 120. The cap may be designed to close once the sensor assembly 110 has been inserted into the housing portion 120. The cap may be attached to the rest of the probing portion 105 via a hinge 820 or other suitable mechanism. In this way, the cap can be opened and closed as needed. In various embodiments, the cap may come completely separate from the remainder of the probing portion 105. In such cases, the cap may be put in place via complementary screw threading, snapping mechanisms, magnets, or via any other suitable mechanism.

In various embodiments, the cap may provide further protection for the sensor assembly and/or for any other components enclosed within the probing portion.

In various embodiments, the optical fibers may constitute a separate or physically detached component from the rest of the sensor assembly 110. For example, in operation, a doctor may insert the optical fibers first, and then may insert the sensor assembly. In various embodiments, an opto coupler (e.g., an optical coupler) may lie between the optical fibers on the one hand and the camera and/or light source on the other hand. The opto coupler may magnify, focus, refract, diverge, concentrate, or otherwise channel light in an appropriate fashion as it goes to and from the optical fibers. For example, the opto coupler may concentrate light from the light source into a narrow beam suitable for traversing the optical fibers.

In various embodiments, the opto coupler is attached to the optical fibers, but not directly to the sensor assembly 110. In various embodiments, the opto coupler is attached to (and/or part of) the sensor assembly, but not directly to the optical fibers. In various embodiments, the opto coupler is a standalone component.

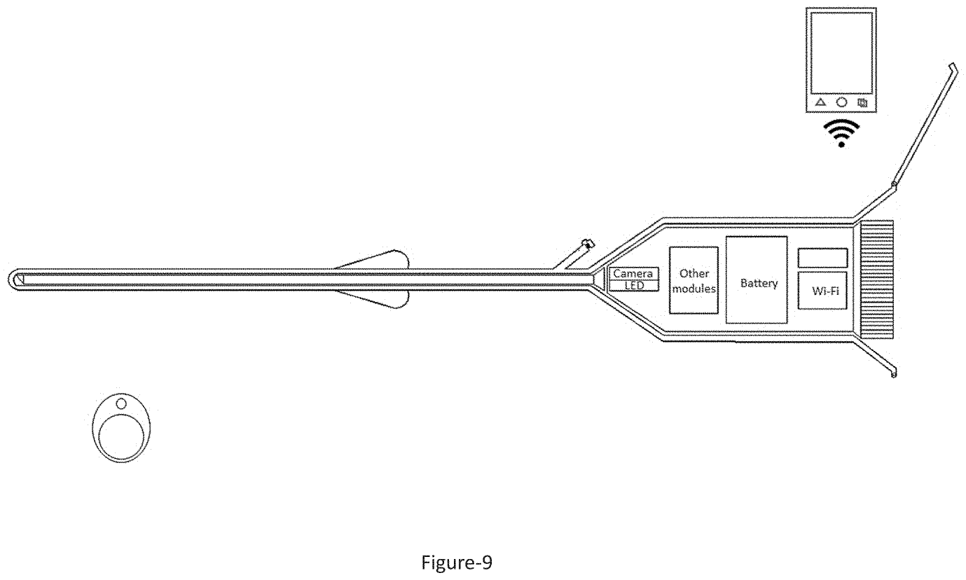

With reference to FIG. 9, a device is shown according to some embodiments. The device may include a saline port. The saline port may lead, via a path, tunnel, tube, channel, etc., to the tip, where it may open into the surrounding area. Thus, the saline port may permit injection of saline water (or other fluid) into the patient. Thus, a path for saline may differ in its construction from the tube containing inserted fiber optics or other re-usable equipment, in that the path for saline may be open at two ends (including one inside the patient), whereas the channel containing re-usable equipment may be open only at one end.

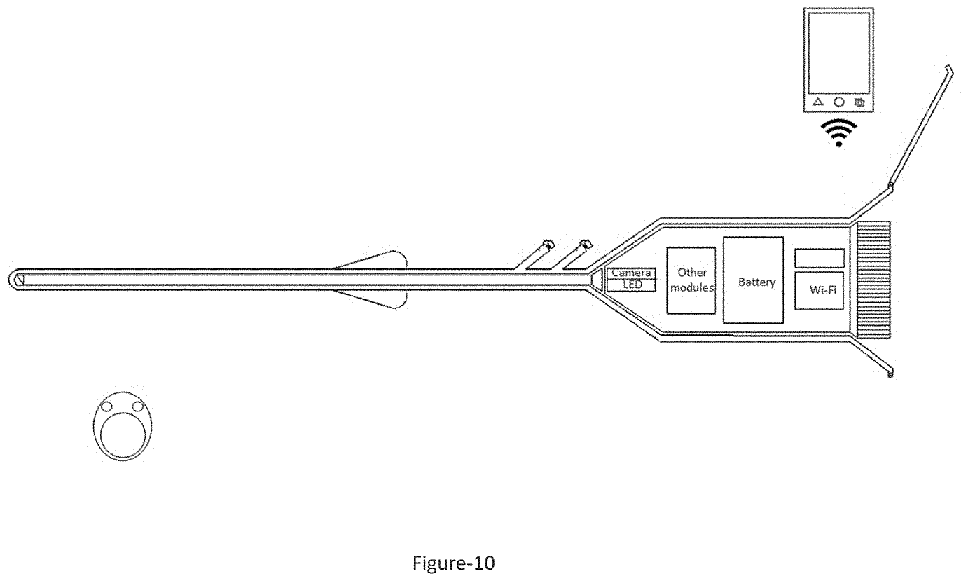

With reference to FIG. 10, a device is shown according to some embodiments. The device includes two saline ports.

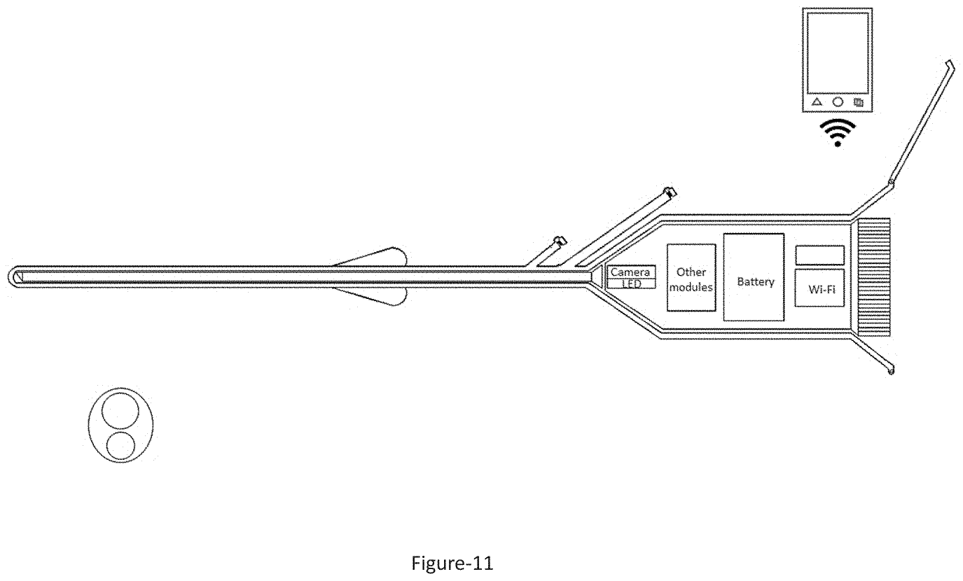

With reference to FIG. 11, a device is shown according to some embodiments. The device includes a saline port and an instrument port. In various embodiments, the saline port and the instrument port lead to the same path in the shaft 115. In various embodiments, the saline port and the instrument port may enter into separate paths to the tip.

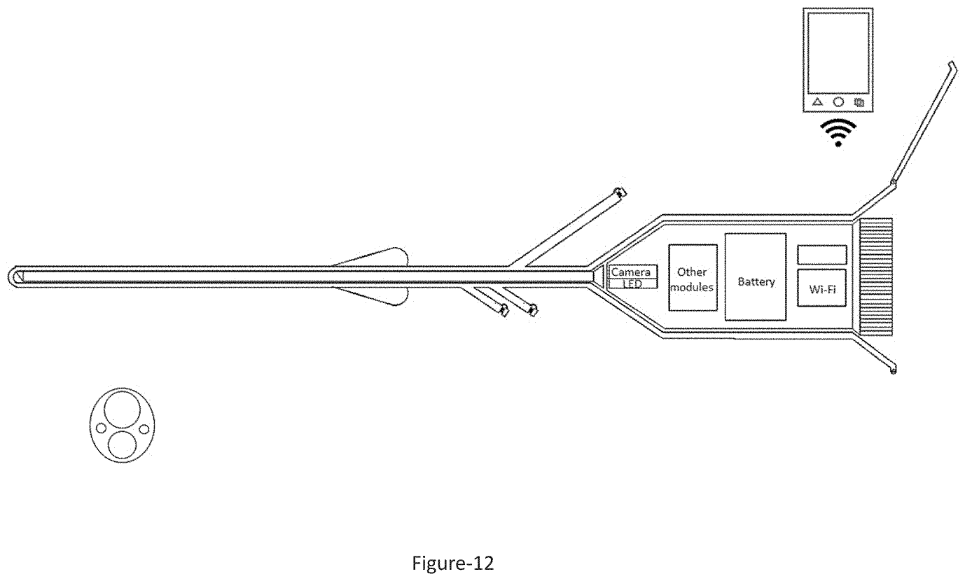

With reference to FIG. 12, a device is shown according to some embodiments. The device includes two saline ports (e.g., for saline injection and suction). The device may further include an instrument port.

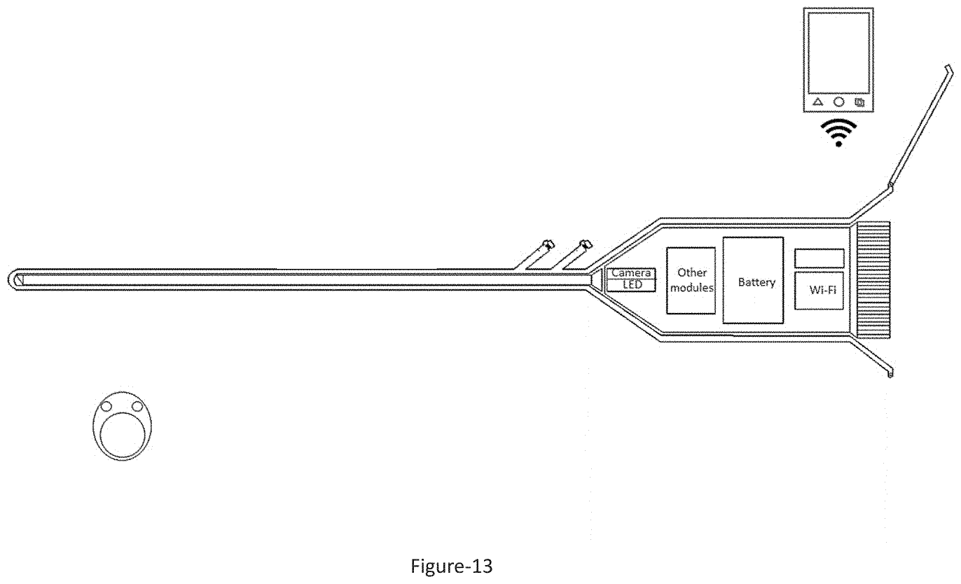

With reference to FIG. 13, a device is shown according to some embodiments. The device includes two saline ports. The device does not include a back-flow stopper.

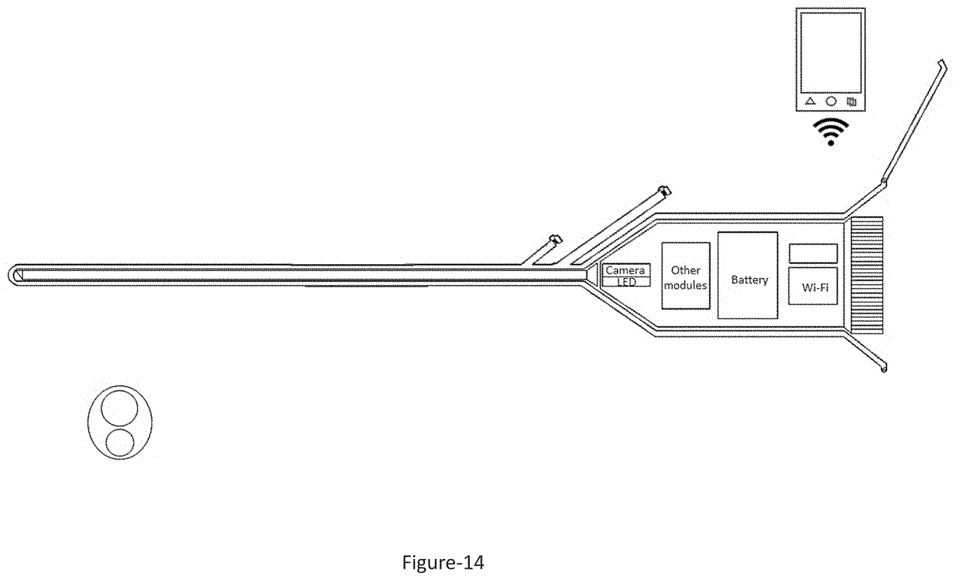

With reference to FIG. 14, a device is shown according to some embodiments. The device includes instrument and saline ports. The device does not include a backflow stopper.

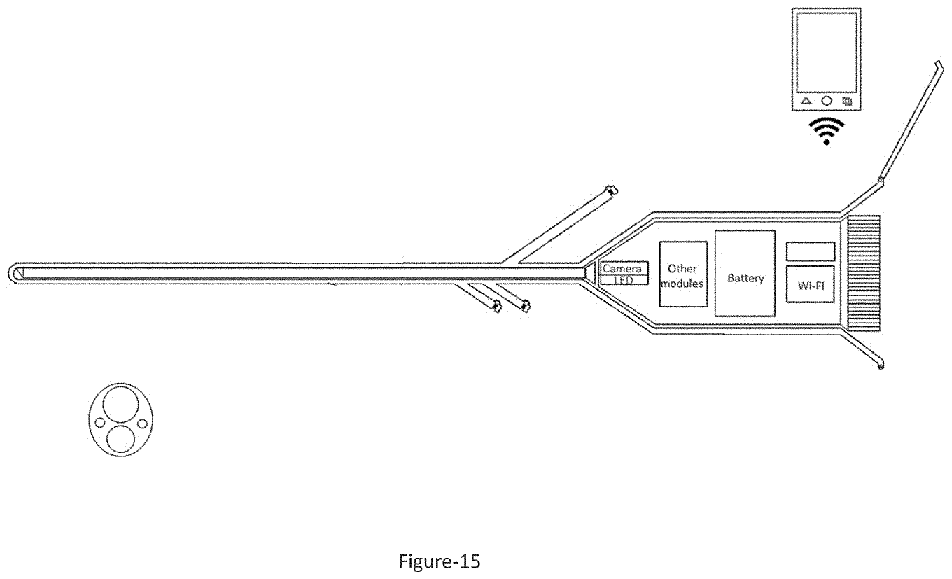

With reference to FIG. 15, a device is shown according to some embodiments. The device includes two saline ports (for saline injection and suction), and an instrument port. The device does not include a backflow stopper.

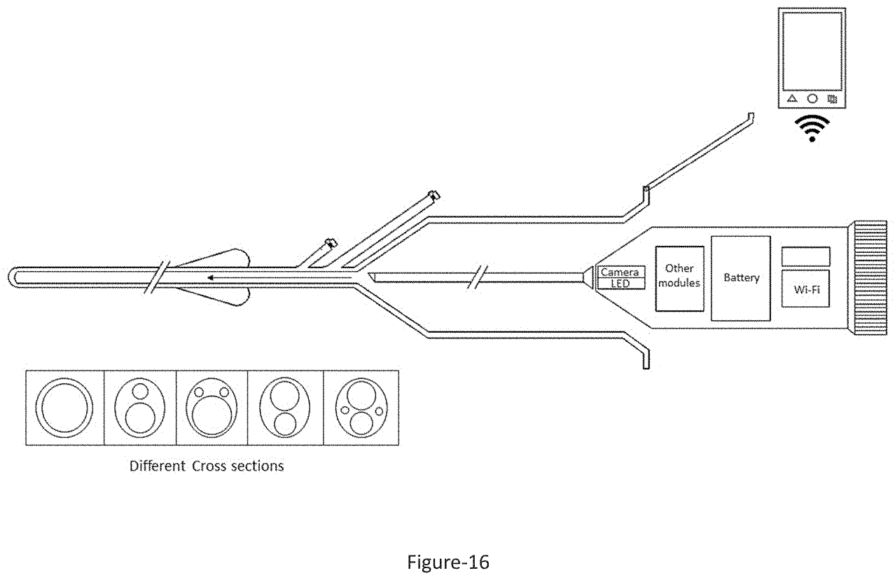

With reference to FIG. 16, a device is shown according to some embodiments. The optical fibers and the sensor assembly are seen prior to complete insertion into the probing portion 105. In various embodiments, data can be streamed in to an external device (e.g., tablet) provided with the product by means of Wi-Fi or via any other means.

Electronic Components Inserted into the Shaft

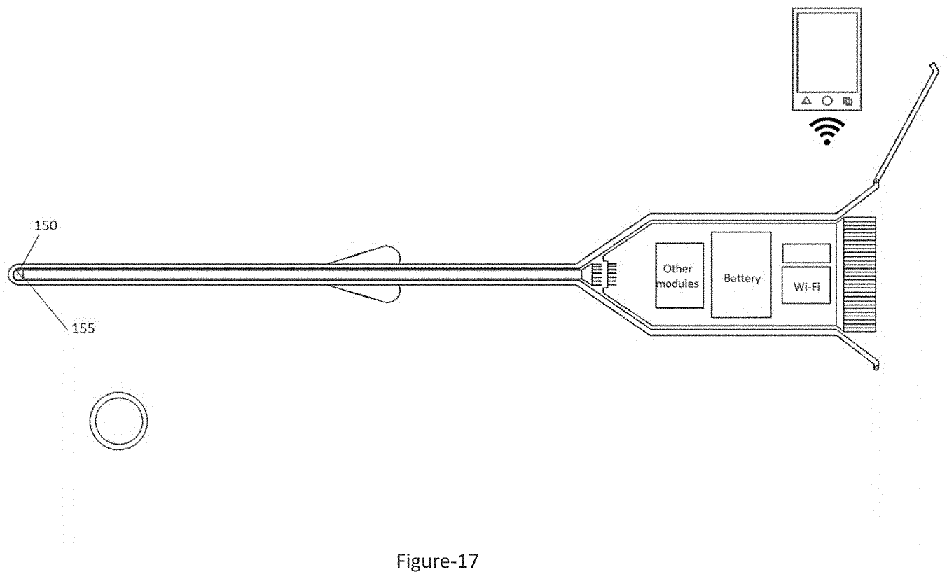

FIGS. 17-25 depict various embodiments in which one or more electronic components are inserted into the shaft 115 of probing portion 105. The electronic components may include a camera. The electronic components may include a light source. The electronic components may be inserted into the shaft 115 such that they reach a point at or proximate to the tip 125 of the shaft 115. The electrical components may avoid direct contact with the patient and may thereby be reused. As will be appreciated, the electronic components may be inserted with supporting structures (e.g., rods). The electronic components may also be connected with wires, cables, etc., in order to transmit data and/or power to and from the sensor assembly 110.

In various embodiments, the camera may feature the Omnivision-OV6946.

With reference to FIG. 17, a device is shown according to some embodiments. Various embodiments include a disposable sheath (e.g., probing portion 105) and reusable camera module. The camera module may capture images. The camera module may capture video. In various embodiments, the device may be used for inspecting the uterus. In various embodiments, the device may be used for inspecting other body parts. In various embodiments, the camera 155 and light source 150 are located at the end of a rod that is to be inserted into the shaft 115 of the probing portion 105. The camera and light source may thereby reach a location at or near the tip 125 of probing portion 105.

In various embodiments, the camera module is a separate unit from the sensor assembly 110. The camera module may connect to the sensor assembly via plug or via any other suitable means. Also, in various embodiments, the camera module can be removed separately if any damage happened due to more cycles of usage. In various embodiments, the camera module may be replaced while the sensor assembly remains the same. In various embodiments, the sheath (e.g., probing portion 105) can be disposed after each use. The camera and other modules can be reused.

In various embodiments, the camera module and the sensor assembly 110 are a single unit. The sensor assembly may include a long, projecting rod designed for insertion into the shaft 115 of the probing portion 105. The camera may lie at the tip of the rod. The light source may lie at the tip of the rod.

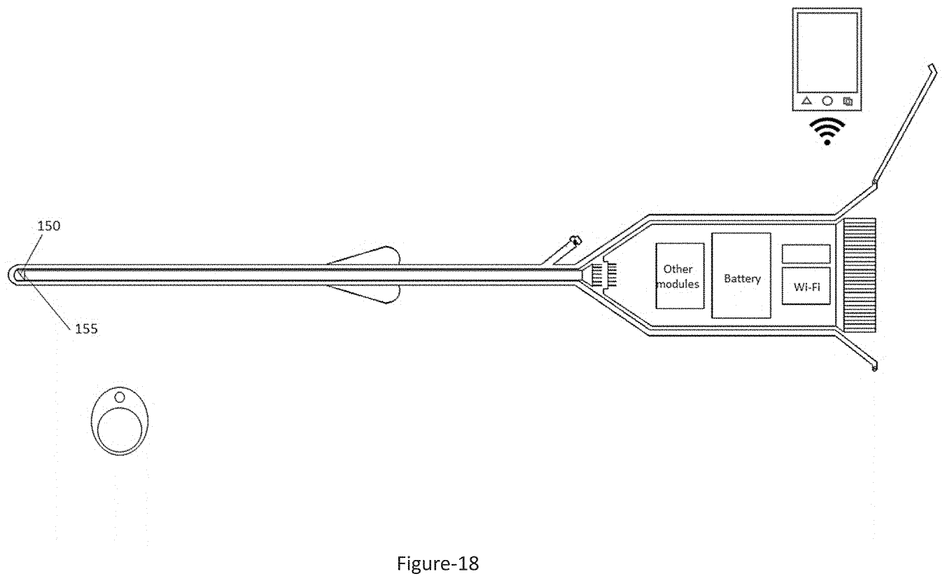

With reference to FIG. 18, a device is shown according to some embodiments. The device includes a saline port and saline path.

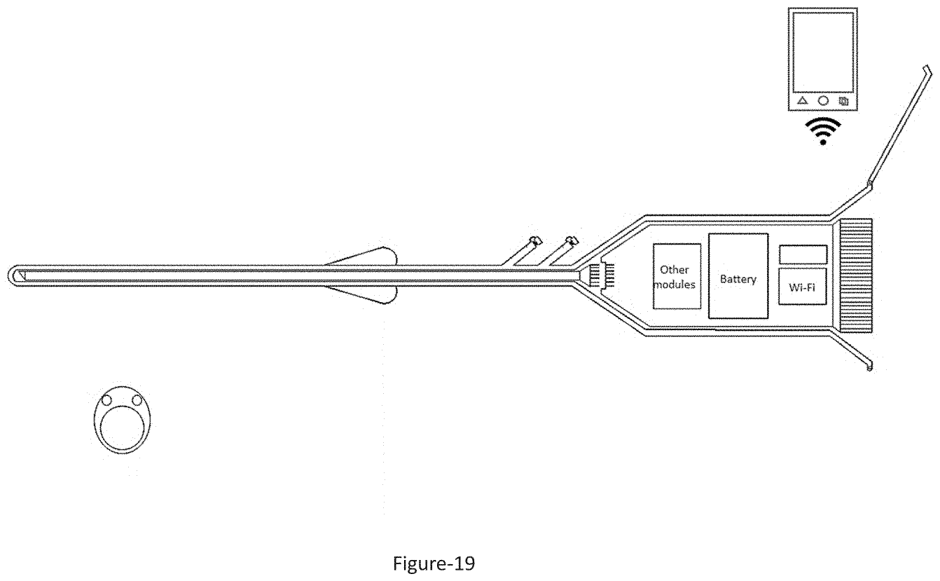

With reference to FIG. 19, a device is shown according to some embodiments. The device includes two saline ports (for saline injection and suction).

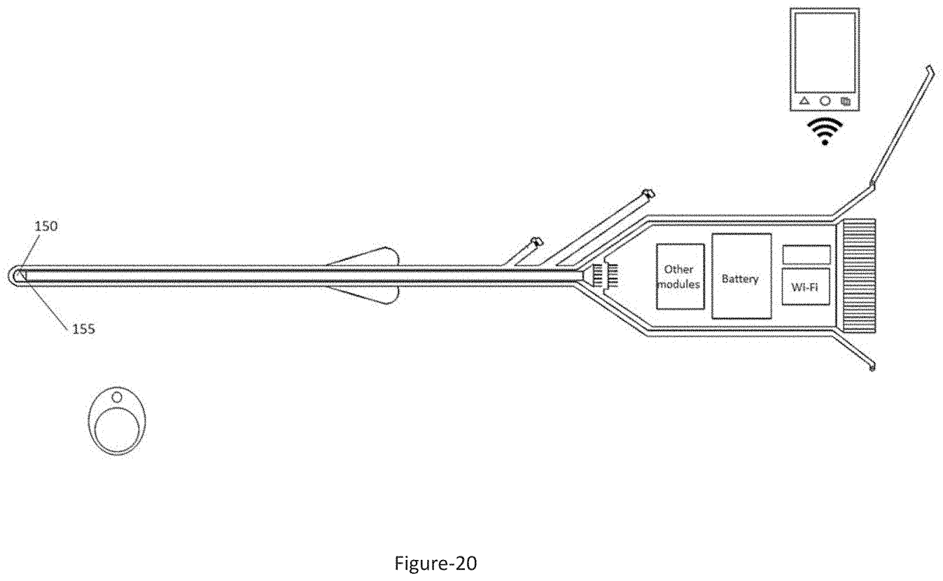

With reference to FIG. 20, a device is shown according to some embodiments. The device includes a saline port and an instrument port. In various embodiments, the saline port and the instrument port lead to the same path in the shaft 115. In various embodiments, the saline port and the instrument port may enter into separate paths to the tip.

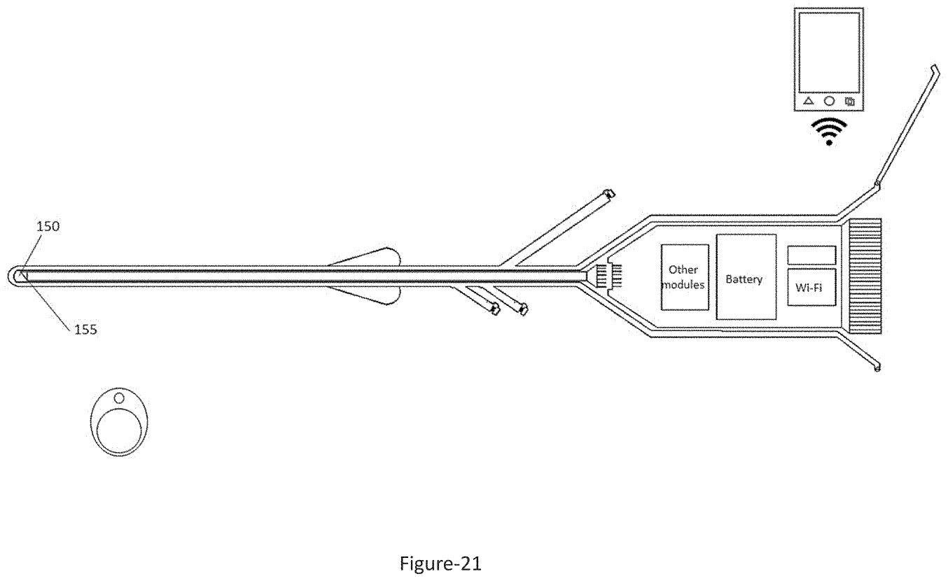

With reference to FIG. 21, a device is shown according to some embodiments. The device includes two saline ports (i.e., for saline injection and suction). The device may further include an instrument port.

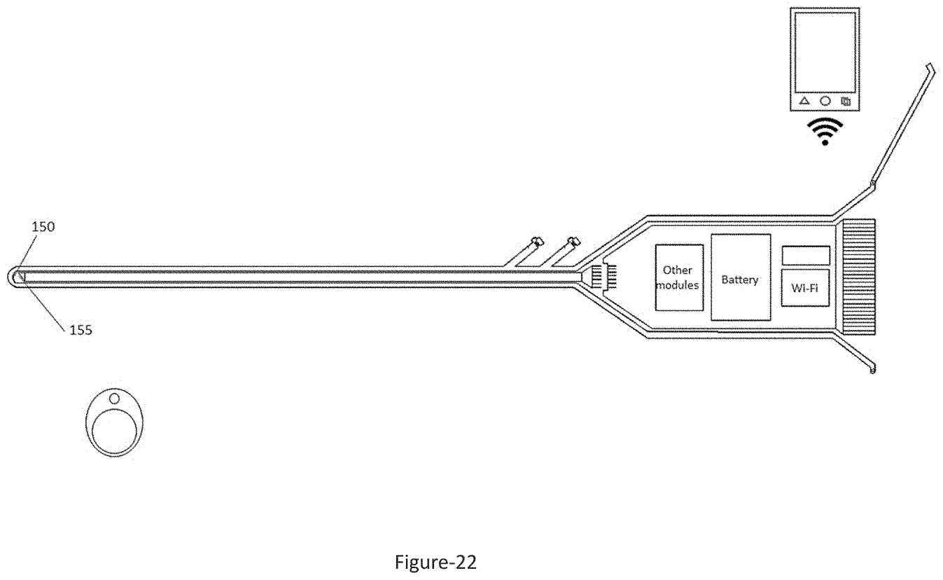

With reference to FIG. 22, a device is shown according to some embodiments. The device includes two saline ports. The device does not include a back-flow stopper.

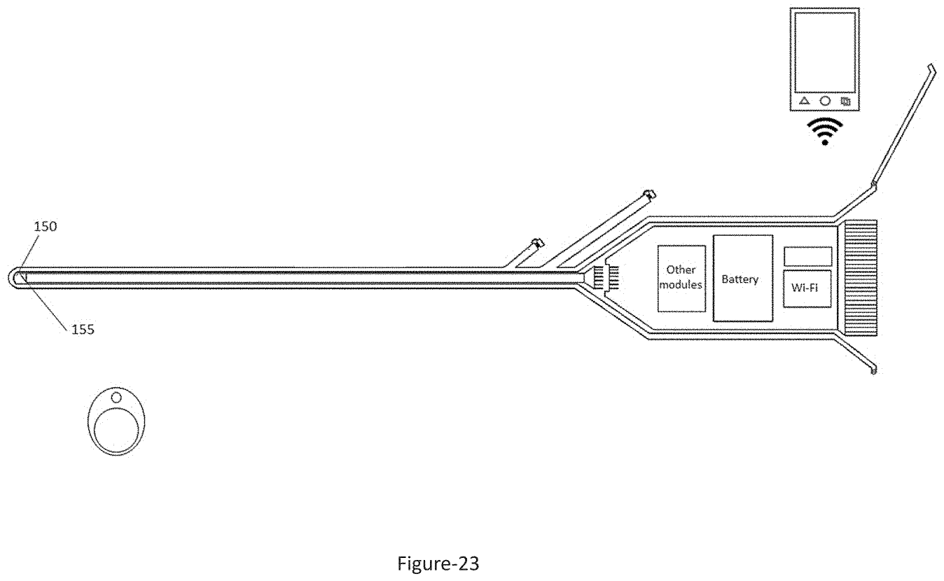

With reference to FIG. 23, a device is shown according to some embodiments. The device includes instrument and saline ports. The device does not include a backflow stopper.

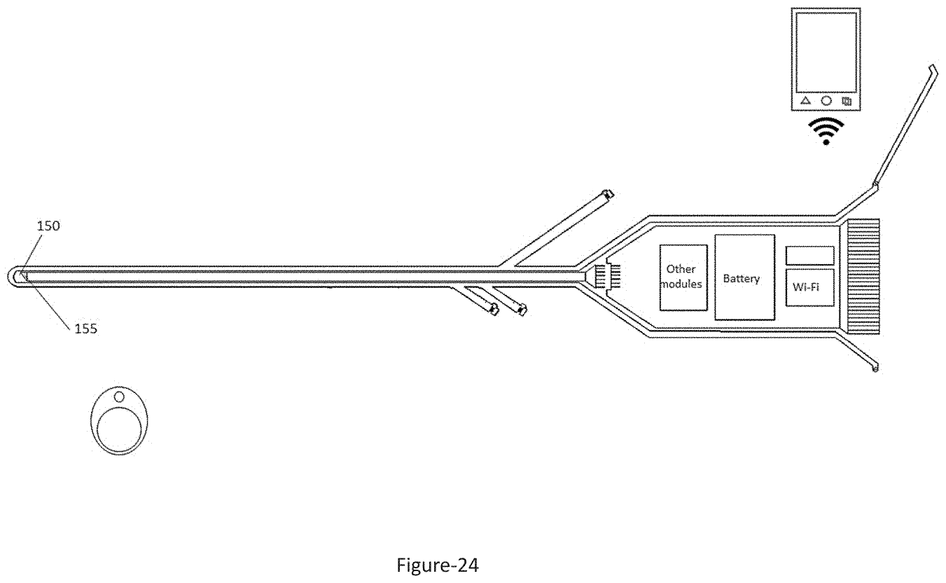

With reference to FIG. 24, a device is shown according to some embodiments. The device includes two saline ports (for saline injection and suction), and an instrument port. The device does not include backflow stopper.

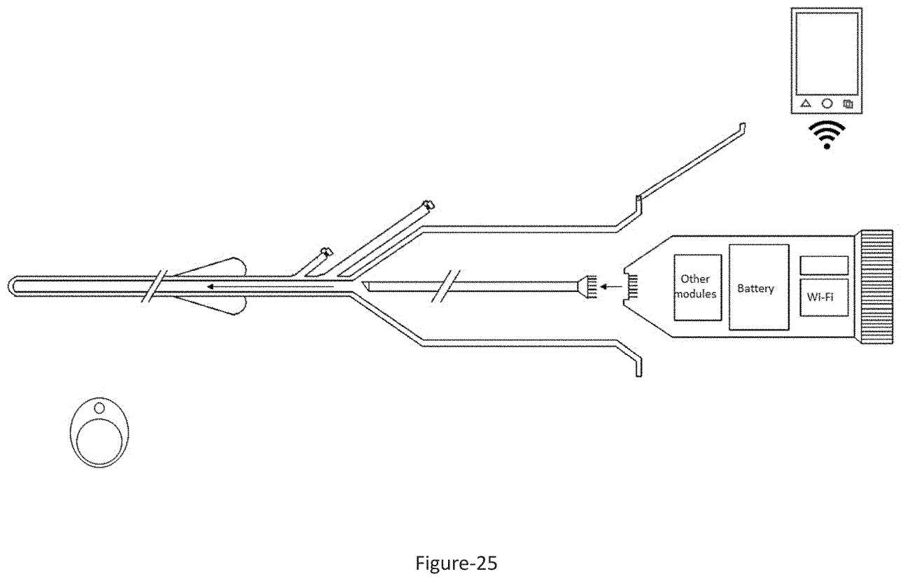

With reference to FIG. 25, a device is shown according to some embodiments. The optical fibers and the sensor assembly are seen prior to complete insertion into the probing portion 105. In various embodiments, data can be streamed in to an external device (e.g., tablet) provided with the product by means of Wi-Fi or via any other means.

With reference to FIG. 26, a block diagram of a device is shown, according to some embodiments. According to various embodiments, a device includes a CMOS Camera module, a light-emitting diode, a video analog to digital converter, an electrical adapter (e.g., a 5-volt adapter), a power supply, a battery charger, a processor (e.g., an ARM 9), a digital signal processor, memory (e.g., random access memory; e.g., read-only memory), a keypad, a video display, and a communications port (e.g., for Wi-Fi).

### Backflow Stopper

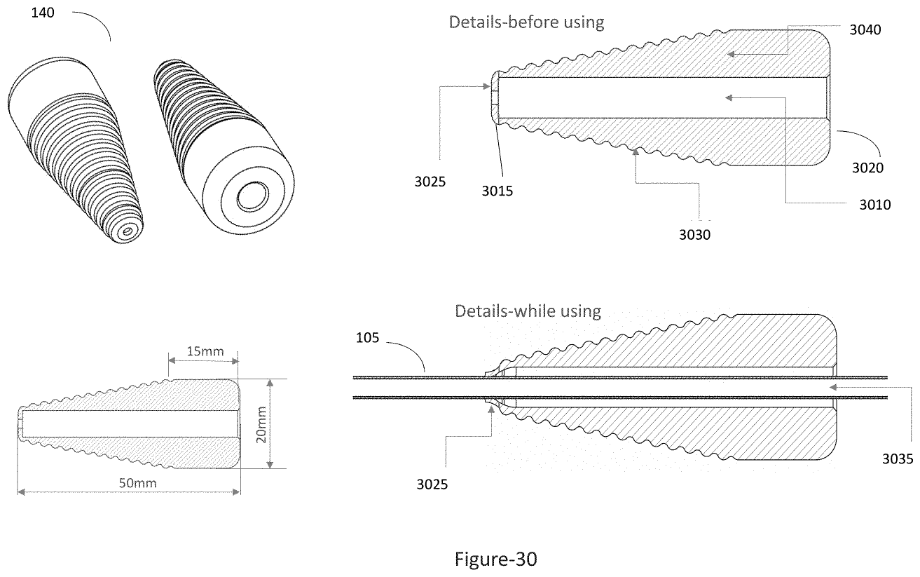

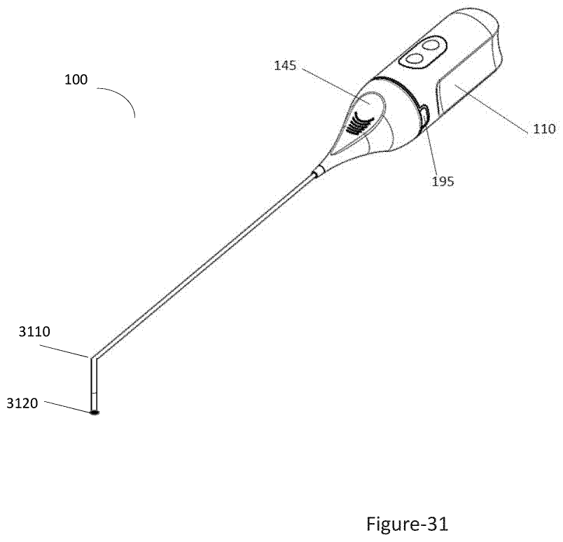

With reference to FIG. 30, a device component, backflow stopper 140, is shown according to some embodiments. Various views are provided, including views from outside, and cross-sectional views.

The backflow stopper may take the overall shape of a hollowed cylinder with gradually tapering outer diameter. The overall shape may appear at least partially conical. The overall shape may also appear at least partially cylindrical. The backflow stopper may be substantially cylindrically symmetrical about a central axis.

In composition (e.g., at annular portion 3040), the backflow stopper may be made from any suitable material or combination of materials. These may include hard bio compatible plastic. These may include PVC (Polyvinyl Chloride), PES (Polyether sulfone), PTFE (Polytetrafluoroethylene), PE (Polyethylene), PEEK (Polyetheretherketone), PS (Polysulfone), PC (Polycarbonate), and/or FEP (Fluorinated Ethylene Propylene). This material may form the main part of the backflow stopper, creating the tapered, cylindrical ring. As will be appreciated, the backflow stopper may be made of two or more materials at once.

In various embodiments, a hole or channel 3010 runs through the center of the backflow stopper 140, aligned with the axis of the backflow stopper. The hole or channel may be designed to admit the probing portion 105 (or a sheath or disposable sheath of probing portion). In some embodiments, the hole is 6mm in diameter. Thus, the hole may admit probing portions of up to 6mm in diameter. As will be appreciated, various embodiments contemplate that holes of other diameters may be used (e.g., holes of 5 mm or 7 mm diameter). Such holes would put different constraints on the diameter of a probing portion (e.g., would allow probing portions of up to 5 mm or 7 mm in diameter, respectively). In some embodiments, the hole 3010 may be substantially cylindrical with circular cross-section. However, in various embodiments, holes of other geometries may be used.

In various embodiments, the probing portion 105 and/or the sheath placed through the hole 3010 may itself be hollow (e.g., with channel(s) and/or lumen(s) 3035), and may admit fluid, forceps and/or any other material or device, as described herein. Accordingly, such fluid, forceps etc., may pass through the hole 3010 in the backflow stopper 140.

For the sake of discussion, the respective ends of the backflow stopper 140 may be referred to as the “narrow end” 3015, and the “wide end” 3020. As will be appreciated, these terms are for discussion purposes only and are not intended to limit or constrain the actual sizes or diameters of the backflow stopper.

In various embodiments, at its narrow end, the backflow stopper may include a membrane 3025 that covers or partially covers the end of the hole/channel 3010. The membrane may be flexible or partially flexible. The membrane may be made of silicone, or of any other suitable material. The membrane itself may take an annular shape, and may include a central hole. In some embodiments, the central hole may have a diameter of 2 mm. In some embodiments, the central hole may have other dimensions. In some embodiments, the central hole of the membrane may be substantially smaller than the hole 3010. In some embodiments, the membrane may have no hole initially. The membrane may be designed to be punctured by the probing portion 105 as it is admitted into the backflow stopper 140, as described below.

In various embodiments, at its wide end 3020, the backflow stopper may have a membrane that covers or partially covers the end of hole/channel 3010. In various embodiments, a membrane may be situated within hole/channel 3010, and may completely or partially divide the hole/channel 3010 into two parts. In various embodiments, any barrier or material may wholly or partially block the hole/channel 3010. Such a barrier or material may be sufficiently soft to allow admission or penetration by the probing portion 105.

In various embodiments, the backflow stopper 140 may admit a probing portion 105 with a diameter that is less than the diameter of the hole 3010. Thus, the probing portion may not necessarily fit snugly within the backflow stopper. However, when the probing portion is inserted into the backflow stopper, it may puncture the membrane 3025 (or any other membrane or obstruction), and thus expand the central hole within the membrane 3025 to just the diameter of the probing portion. Thus, the probing portion may fit snugly at least within the punctured membrane. The snug fit may serve to eliminate or reduce backflow of fluid through the hole/channel 3010. It may also reduce or eliminate slippage of the probing portion 105 with respect to the backflow stopper 140.

In various embodiments, at its widest point with respect to a plane perpendicular to the central axis (e.g., at the wide end 3020), the backflow stopper may have a diameter of 20 mm. In various embodiments, at its narrowest point with respect to a plane perpendicular to the central axis (e.g., at the narrow end 3015), the backflow stopper may have a diameter of 7 mm. As will be appreciated, various embodiments contemplate a backflow stopper with other dimensions as well.

In various embodiments, the total length of the backflow stopper may be 50 mm. Over a first portion of this length (e.g., 15 mm), the backflow stopper may maintain a constant diameter (e.g., the same diameter it was at its widest point; e.g., 20 mm). Over a second portion of its length (e.g., 35 mm), the backflow stopper may have a substantially tapering diameter, such that its diameter gradually decreases until reaching the narrow end 3015 of the backflow stopper. At this point, the backflow stopper may be 7 mm in diameter. As will be appreciated, various embodiments contemplate a backflow stopper with other dimensions as well.

In various embodiments, a portion of the backflow stopper (e.g., the tapering portion 3030) may take on a grooved, wavy, bumpy, serrated, and/or jagged shape. The figure provides an illustration of this shape according to various embodiments, although it will appreciated that various embodiments contemplate other possible shapes as well (e.g., shapes with more jagged corners). This shape may allow the backflow stopper to “catch” more readily and/or securely within a desired part of the body (e.g., within the cervix).

### Examplary Names

A device according to various embodiments may be named “Hysto-view” “Endo-view”, “Cysto view”, or any similar name, or any other name.

### Sheath Providing Complete Enclosure

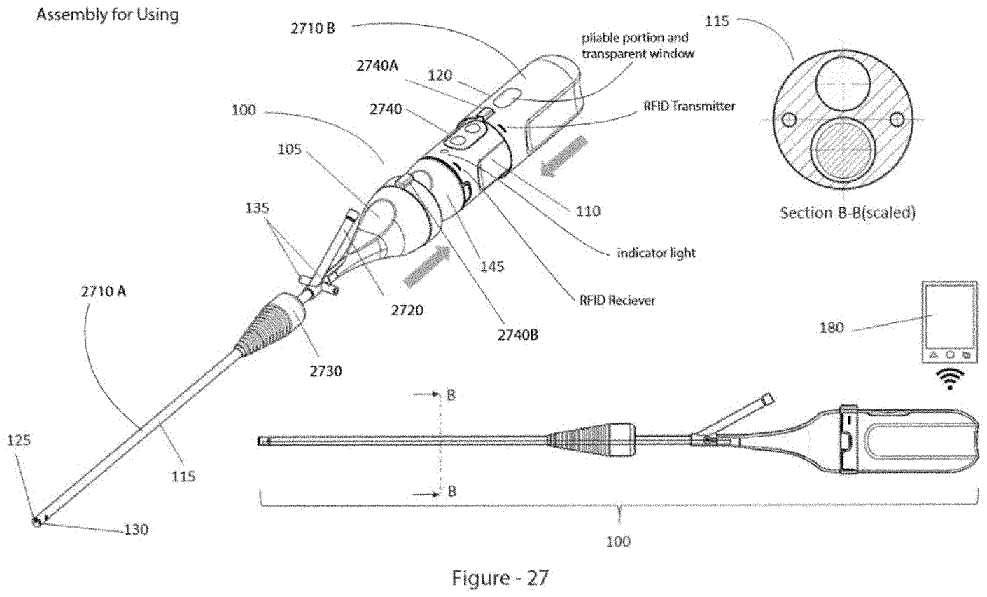

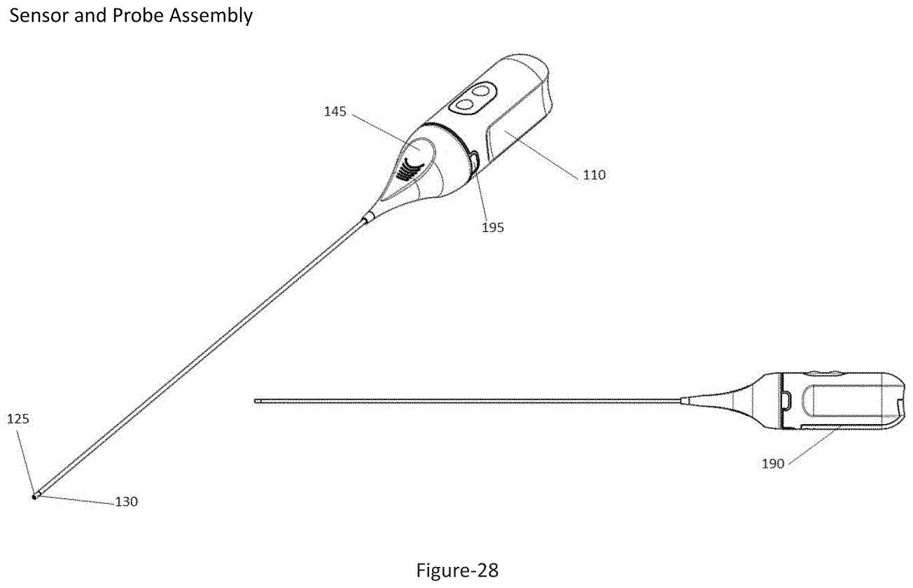

With reference to FIG. 27, a device 100 is shown according to some embodiments. The device includes a probing portion and a main body. The main body may include a sensor assembly and/or any other electronics. The main body may include battery, battery pack, processor, memory, storage, electronic wiring and/or any other suitable components. In various embodiments, the probing portion and main body may form a single unit. In various embodiments, the probing portion may be attachable and detachable from the main body.

With continuing reference to FIG. 27, a sheath is shown according to some embodiments. In various embodiments, the sheath may fit over the device in one or more sections. The sheath may serve to completely cover the device. The sheath may thereby isolate the device from the surrounding environment, and may protect the device from contamination (e.g., from pathogens hosted by a patient during a procedure).

In various embodiments, a sheath covers the device in two separate sections which then join together to form a complete covering. In various embodiments, a first section of the sheath 2710A covers the probing portion, and a second section of the sheath 2710B covers the main body. The first section and the second section of the sheath then join together to form a complete covering. The first section and the second section may be joined together via any suitable means, including a snapping mechanism, locking mechanism, adhesive, screw mechanism, etc.

In various embodiments, the sheath may comprise more than two sections (e.g., three sections, e.g., four sections, etc.) Each of the sections may be joinable so as to completely enclose the device.

In various embodiments, the sheath may serve other functions in addition to serving as a covering or barrier to the environment. The sheath may include one or more functional components.

In various embodiments, a sheath includes one or more channels or lumens. A channel may allow transmission of saline or other solution through a portion of the sheath; the admission of forceps through a portion of the sheath, and/or a channel may permit any other function. In various embodiments, a channel admits a portion of the device itself (e.g., the probing portion of the device). The channel may thereby endow the sheath with its function of covering/enclosing the device.

In various embodiments, a sheath includes one or more ports (e.g., port 2720). The ports may provide a connection between the outside and one or more channels or lumens. For example, a port may allow saline solution to be inserted into the lumens from an external reservoir. For example, a port may allow the admission of forceps into a channel of the sheath.

A sheath may include a backflow stopper 2730. The backflow stopper may encircle a portion of the sheath. In various embodiments, the backflow stopper may be fixed to the sheath. In various embodiments, the backflow stopper may be slidable along the length of the sheath (e.g., along the length of a portion of the sheath covering the probing portion). The backflow stopper may include concentric grooves/serrations that are designed to wedge inside a bodily opening, regardless of the size of the opening. The backflow stopper may reduce or prevent leakage of fluid from inside the body cavity.

In various embodiments, it may be desirable that an operator of a device have access to certain actuators, control buttons, knobs, dials, switches, etc. (e.g., 2740), associated with the device. Such actuators may be built into the main body, in various embodiments. The actuators may allow the operator to: turn the device on or off, take a picture, turn a light source on or off, adjust the brightness of a light source, initiate video capture, terminate video capture, initiate streaming, terminate streaming, change the resolution of an image, and/or any other suitable functionality.

In various embodiments, the sheath permits activation of one or more actuators through the sheath. The sheath may be thin, pliable, and/or flexible to permit pressing of buttons and/or activation of any other controllers through the sheath. The sheath may be sufficiently thin to allow use of touch controls through the sheath. In various embodiments, the sheath may be transparent, transparent over a portion of its surface area, translucent, and/or partially transparent. This may allow an operator to see actuators and/or markings on the device through the sheath.

### Snap Mechanism

In various embodiments, two portions of the sheath may fit together and may thereby completely enclose the endoscopic device inside. In various embodiments, each of the two sheath portions may have complementary circular openings, with one slightly smaller than the other. In various embodiments, complementary screw threads may allow one sheath portion to partially twist or screw inside the other portion. In various embodiments, complementary grooves may allow one sheath portion to fit inside the other at a preferred orientation. For example, a protruding piece from the outside of one sheath portion may fit inside a complementarily shaped cavity portion on the outside of the other sheath portion. These may allow the two sheath portions to join in a preferred orientation (e.g., at a preferred angle with respect to one another).

In various embodiments, one or more snapping or locking mechanisms may serve to fix the two sheath portions together. The mechanisms may prevent them from coming apart. The mechanisms may also prevent the sheath portions from changing orientation with respect to one another (e.g., from rotating with respect to one another). Exemplary interlocking portions of a snap mechanism are shown with reference numerals 2740A and 2740B.

In various embodiments, external markings may provide a visual indicator for when two portions of a sheath are in the correct orientation with respect to one another. For example, respective line segments on each of the sheath portions may together form a continuous line when the sheath portions are joined and oriented correctly. As will be appreciated any other suitable image, pattern, marking, etc., is similarly contemplated according to various embodiments.

In various embodiments, a locking mechanism for two sheath portions may include a release mechanism. The release mechanism may allow the two sheath portions to be separated when the mechanism is activated (e.g., when a button or lever is pressed). For example, once a procedure has concluded, it may be desirable to separate the sheath portions so that the internal device can be recovered with the sheath mechanisms are then discarded.

Reference numerals not specifically described with respect to FIG. 27 may represent analogous components to those described by like numerals with respect to FIG. 1. However, it will be appreciated that FIG. 27 depicts a sheath that is covering device 100. Therefore, it will be understood that, in various embodiments, reference numerals refer to an analogous component making up the sheath, making up the device under the sheath, or both.

### Sheath Effects Operation of Device

In various embodiments, the device 100 may only function when the sheath is on. This may ensure that an operator may not inadvertently proceed with a procedure until the device has been safely enclosed in the sterile sheath, and thus risk of contamination has been minimized.

In various embodiments, the device 100 may detect when the sheath is on. Detection may occur via radio-frequency identification (RFID), near-field communication (NFC), Bluetooth, Wi-Fi, or via any other communication means, or via any other means. For example, the sheath may include an RFID transmitter, and the device may include an RFID receiver that is configured to receive signals from the RFID transmitter of the sheath, where such signals may indicate the presence of the sheath and/or proper configuration of the sheath.

In various embodiments, use of an “on” button on the device, or other actuation means, may be dependent on the presence of the sheath and/or may be dependent on the proper configuration of the sheath. In various embodiments, an operator of the device may press an “on” button of the device through the sheath. However, in various embodiments, the sheath may have a pliable portion that must be aligned (e.g., over) the button on the device in order that the operator can press the button through the sheath. If the sheath is not configured correctly, the pliable portion of the sheath will not be aligned with the button, and the operator may be unable to turn the device on.

In various embodiments, the device may turn on automatically when the sheath is placed on it. In various embodiments, the device may turn on automatically when the sheath is placed on it and configured properly (e.g., when two parts of the sheath are appropriately latched into place).

In various embodiments, certain functions of the device may be dependent upon the presence of the sheath, and certain functions may not. For example, the device may only capture video when the sheath is on. However, the device may upload data (e.g., to an external server) even when the sheath is not present.

In various embodiments, one or more indicators (e.g., LED lights) on the device and/or on the sheath may indicate when the sheath is in its proper place (e.g., with respect to the device) and/or in the proper configuration. For example, an LED may show green when the sheath is in its proper place, but red when the sheath is not. In various embodiments, an indicator may be audio.

In various embodiments, a warning signal or indicator is provided if the sheath is off and/or is taken off. For example, a red LED might be activated, or a warning chime might play. In various embodiments, a warning signal is provided if the device is activated and/or used when the sheath is not properly configured (e.g., when the sheath is not on; e.g., when the sheath is not put on properly).

In various embodiments, if the device is used without the sheath in its proper configuration, then a warning or other indication of such use may be placed into the patient's record. For example, the device may upload the warning, along with any data from the procedure, to the patient's record. In this way, for example, if there is an infection or other complication resulting from the procedure, the patient's record will show one potential cause of such complication (i.e., use of the device without proper protection by the sheath).

### Luminescence

In various embodiments, one or more buttons, actuators, etc., on the device 100 may glow in the dark. This may allow the operator to find the buttons in the dark. This may also allow the buttons to be more visible without draining power from the device. In various embodiments, buttons, actuators, etc., (or portions thereof) may be made of (or incorporate) a luminescent material additive along with the button material.

### Face Detection

In various embodiments, the device, the camera, the camera module, and/or any component or software module may be capable of performing face detection. Face detection may occur in images and/or in a video feed captured by the device. When a face is positively detected (or detected within a certain confidence level), then one or more steps may be taken to avoid permanent recording of the face. This may allow a patient's privacy (or another party's privacy) to be maintained.

In various embodiments, when a face is detected, corresponding video may be deleted and/or simply not stored to a permanent record. In various embodiments, when a face is detected, the face may be blurred, blacked-out, or otherwise rendered unidentifiable before such image or video is stored in a permanent record.

### Dual Tone LED

In various embodiments, the device 100 may employ a dual tone light emitting diode (LED). The dual tone LED may be situated at the tip of the device 100. The dual tone LED may thereby illuminate the cavity which is the subject of the procedure. The dual tone LED may provide lighting that is similar to natural lighting, and may thereby provide a better video and/or image.

### Voice Commands

In various embodiments, an operator may use voice commands to navigate a user interface (UI). The UI may include the UI of a viewing device (e.g., of an external viewing device; e.g. of an iPad). In this way, the operator may avoid touching a surface with his/her hands and potentially contaminating his hands.

### Attachment of Probing Portion to Main Body

In various embodiments, the device may comprise two or more separate units that can be attached together. In various embodiments a first unit of the device may comprise a probing portion. The probing portion may include a long narrow arm for insertion into a body cavity, for holding an imaging device, for holding a light source, for transmitting saline, and/or for admitting forceps into the body cavity, etc. In various embodiments, a second unit of the device may comprise a sensor assembly, electronics, enclosure for the electronics, place to grip by an operator, actuators, and/or other components. In various embodiments, the second unit may be referred to as a “main body”, “handheld body”, or with similar terminology.

In various embodiments, it may be desirable that the first unit (e.g., probing portion) be detachable from the second unit (e.g., main body) for one or more reasons. In the event that the first unit might become damaged, contaminated, or otherwise compromised, the first unit may be discarded, while the second unit can be maintained. This may save on the expense of a total replacement of the entire device. In various embodiments, the separation of the first and second units may facilitate more compact and/or flexible storage, docking, charging, etc. for the device.

In various embodiments, the first unit (e.g., probing portion) attaches to the second unit (e.g., main body), at least in part via Pogo pins. These may include spring-loaded pins that facilitate electrical and/or mechanical contact between two components. The use of pogo pins may avoid drawbacks of other means of electrical connection. For example, with traditional male/female electronic connectors, the female portion may include holes that can potentially admit fluid or other contaminants. Further, the male pins of electronic connectors may become bent, rendering the connectors unusable. The use of Pogo pins may reduce the risks of contaminants and/or bent pins, according to some embodiments.

In various embodiments, the first unit may be electrically connected to the second unit (e.g., in addition to and/or in conjunction with any mechanical connection). The electronic connection may allow transmission of power (e.g., transmission of power to LED's, camera units, etc.), transmission of control signals (e.g., commands to take a picture, commands to adjust light color or intensity), transmission of data (e.g., images, video, etc.), and/or transmission of any other signals between the first and second units.

### Charging Unit

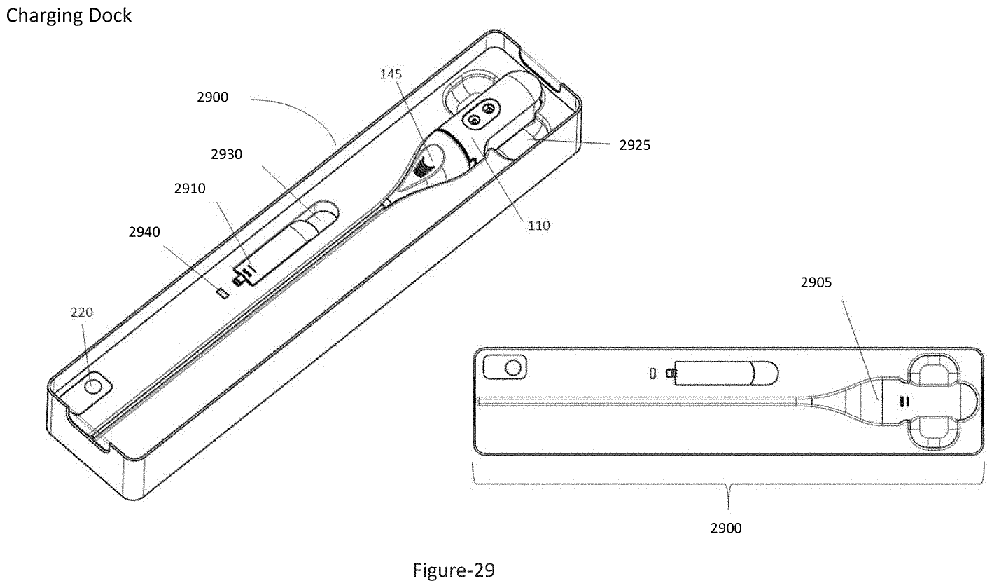

Reference is now made to FIG. 29. Various embodiments including a charging dock 2900. The dock may allow for simultaneous docking and/or charging of a device 100 and also of a backup or alternate battery source 2910. The dock may include a first cavity 2905 in the shape of the device (as depicted in FIG. 29, the first cavity already contains the device). The first cavity may include additional space or spaces 2925 permitting the insertion of the operator's fingers in order to grasp the device when placing it in the dock or removing it from the dock.

The dock may include a second cavity 2930 in the shape of a backup battery pack. In various embodiments, the backup battery pack may be swappable into the main body of the device. In this way, if the battery within the device becomes depleted, the operator may swap in a fresh battery and avoid waiting for the current battery to recharge. Meanwhile, the depleted battery (or battery pack) can be placed in the second cavity 2930 and begin charging so as to be ready if the then fresh battery later becomes depleted.

As will be appreciated, cavities in the docking station may take other shapes and need not necessarily conform precisely to the shapes of the device and/or to the shapes of any backup battery.

The cavities may include electrical interfaces which come into contact and/or proximity to the device or battery pack placed inside. Thus, for example, when the device is placed inside cavity 2905, the device may be situated or oriented so as to have an electrical contact or charging point in contact or proximity to a complementary electrical interface on the docking station. The device may then draw power through the electrical interface in order to recharge.

In various embodiments, an electrical interface may include an interface using direct contact, or a contactless interface (e.g., an interface using inductive charging).

In various embodiments, the electrical interface may also permit data transmission. The device may thereby download data such as recorded images or videos from a procedure.

In various embodiments, the docking station may include internal storage, memory, and/or other electronics. The docking station may communicate with the endoscopic device 100 and may transmit or receive data from it.

In various embodiments, the docking station may itself include a built-in battery, fuel cell, power brick, or other power storage unit. Thus, the docking station may be used to recharge device 100 and/or backup battery 2910 even if the docking station itself is not connected to grid power.

In various embodiments, a built-in battery of the docking station may lie underneath the cavities for receiving the device 100 and backup battery pack 2910.

In various embodiments, the docking station may have an electrical interface (e.g., plug) for connection to grid power (e.g., to AC mains). This may allow the docking station to charge its own built-in battery. This may also or alternatively allow device 100 to draw power directly from the grid.

In various embodiments, the docking station may include one or more indicators showing a charge status (e.g., indicator 2940). An indicator may show the charge status of the built-in battery for the docking station. An indicator may show the charge status for the endoscopic device 100. An indicator may show the charge status for the backup battery 2910. Various embodiments may include one, all, or any combination of the aforementioned indicators.

In various embodiments, the indicators may be LED lights. The lights may be colored, or multi-colored. In various embodiments, the indicators may take any other suitable form.

In various embodiments, an LED indicator emits green light to indicate full charge, amber light to indicate partial or mid-range charge, and a red light to indicate low charge. As will be appreciated, a single LED indicator may vary its color as appropriate, or alternatively multiple single-colored LEDs may be used, with the appropriate LED activated based on a current charging status.

In various embodiments, an indicator itself may draw power. Thus, it may be desirable for the indicator to be active for only a limited period of time. In various embodiments, the activity of the indicator is dependent on whether or not the docking station is connected to (e.g., plugged into) grid power. In various embodiments, an indicator becomes active only when requested by a user.

In various embodiments, the dock includes a button or other actuator. When the button is pressed, one or more indicators may show charging status. For example, an indicator LED may emit light for five seconds once the button is pressed, and may then go off. In this way, the LED indicator can minimize its depletion of the battery to which it is connected. A charging status need only be shown when requested by an operator (e.g., when an operator presses the appropriate button on the docking station).

In various embodiments, if the docking station is connected to grid power, an indicator may function in a first fashion, and if the docking station is not connected to grid power, an indicator may function in a second fashion. In various embodiments, if the docking station is connected to grid power, then an indicator light may blink when showing a charging status. If the docking station is not connected, then an indicator light may remain on continuously while showing a status, and may then turn off.

In various embodiments, an indicator light may remain on longer when there is connection to grid power versus when there is no connection to grid power.

In various embodiments, the endoscopic device 100 itself may have an inbuilt indicator to show a level of charge. In various embodiments, the backup battery pack may have an inbuilt indicator to show a level of charge.

In various embodiments, a battery of the endoscopic device 100 may hold a charge for up to eight hours of operation of the device. As will be appreciated, various embodiments contemplate other battery capacities as well.

### Inputs and Data Handling

In various embodiments, during a procedure, an operator may need to rotate the endoscope device 100 about its long axis. This may allow the operator to sweep the camera at the tip of the device in an arc to gain a greater viewing coverage. However, as the operator rotates the device, one or more buttons, actuators, etc. (e.g., buttons on the main body), may change their position relative to the operator's hand (e.g., the hand holding the device). Depending on the extent to which the device is rotated, it may be awkward for the operator to activate or touch one or more buttons, actuators, etc.

In various embodiments, one or more functions of the device occur automatically.

In various embodiments, the device 100 begins recording video automatically when it is turned on. In various embodiments, the device begins taking pictures (e.g., periodically, e.g., one every second) when it is turned on.

In various embodiments, the device 100 may have position sensors, gyroscopes, tilt sensors, etc. to detect its own position, orientation, etc. The device may have contact sensors, temperature sensors, etc. The device may use readings from one or more sensors in order to infer its location (e.g., inside or outside of a body cavity). For example, if the device measures an ambient temperature corresponding to body temperature, it may infer that it is inside a body cavity. Based on its location, the device may take one or more actions, such as capturing film, activating an LED (e.g., an illumination source), taking a picture, or any other action.

In various embodiments, the device may include a microphone. The device may detect audio commands, and may respond to voice commands (e.g., begin filming, etc.).

In various embodiments, the device 100 is in wireless communication with an external device. The external device may receive one or more commands (e.g., via touch, mouse, keyboard, voice, etc.) and may wirelessly transmit such commands to the endoscopic device. In this way, for example, an operator can input commands at a fixed control panel even while the operator rotates the device.

An endoscopic device according to various embodiments has an internal memory card. The memory card may be a micro-sd card. The memory card may be a 64 GB micro-sd card. As the device captures video, the video may be stored on the memory. The video may be simultaneously streamed to an external device (e.g., to an external tablet). In this way, an operator may watch the video live on the external device, and may make decisions, change a field of view, position forceps, etc., based on the video.

In various embodiments, video may be captured at a first bit-rate, resolution, etc., and may be streamed at a second bit-rate, resolution, etc. In various embodiments, the video may be streamed at a lower resolution than that at which it was captured. This may allow the operator to view real-time video feeds without exceeding communication bandwidth, tablet display resolution, and/or any other constraint.

In various embodiments, the internal memory may store video at a higher bit-rate, resolution, etc., than that at which the video was captured. In various embodiments, the internal memory may store uncompressed video.

Following a procedure, an operator may wish to view and/or download higher quality images and/or video than those which he/she was viewing on a display screen during the device. According to various embodiments, images and/or videos (e.g., uncompressed videos) may be downloaded from the device following the procedure. The download may occur via wireless transmission, Wi-Fi, physical cable, or via any other means.

In various embodiments, the endoscope device and/or an external device (e.g., a device in communication with the endoscopic device) may ask the operator if he/she wishes to download data captured from the procedure (e.g., videos; e.g., images). The download may occur to an external device, to the cloud, or to anywhere else. The operator may then signify approval in order to begin the download. In various embodiments, the download may occur automatically without explicit command or approval from the operator.

In various embodiments, if the internal storage capacity of the device is exceeded, then the device may delete stored data. For example, the device may delete old videos or old images. In various embodiments, oldest data is deleted first. In various embodiments, data is deleted automatically after some period of time (e.g., 30 days).

### Sheath and Angled Tip

In various embodiments, the probing portion of a device 100 may have a bend or angle 3110 in an otherwise straight arm. An imaging device (e.g., camera) and/or light source (e.g., LED) may lie at the very tip 3120 of the arm. The bend may therefore serve to point the imaging device and/or light source in a different direction than along the main axis of the probing portion. For example, the center of the camera's viewing cone may form a line that is at an angle to the main axis of the probing portion. This angle may correspond to the angle of bend in the probing portion (e.g., arm of the probing portion). As will be appreciated, in various embodiments, the bend may be instantiated as a sharp corner or gradual curvature.

In various embodiments, the bend in the tip of the probing portion may allow an operator to obtain a more complete view of a body cavity by rotating the entire device (i.e., endoscopic device) about a central axis. This rotation may cause the camera to trace an arc within the body cavity and thereby obtain more viewing coverage. As will be appreciated, were the camera exactly in line with the axis of the device, turning the device about its axis would not increase viewing coverage, but would only change the orientation (i.e., rotate the orientation) with which the same view is captured. The same advantages may apply with respect to the light source as well. Namely, with a light source at the tip of the bent probing portion, rotating the device will sweep the light source in an arc to illuminate a greater field of view.

In various embodiments, the device includes a main body (e.g., handheld body), comprising electronics and one or more finger-operated control actuators. A long, narrow probing arm has a first end attached to the handheld body, and a second end having an imaging device, in which the probing arm is substantially straight but for a single bend which causes the second end to form a first angle with respect to the first end. In various embodiments, the second end may have a light source.

In various embodiments, the angle of the bend in the probing portion may be controllable by the operator. For example, the operator may use a button, slider, or other actuator to vary the bend from zero degrees to as much as 45 degrees. (Various embodiments contemplate other ranges of angles as well). The bend may be controlled via an adjustable hinge, a piezoelectric material that can expand or contract one side of the probing arm (thereby causing it to bend), a fiber or pulley to preferentially pull one side of the probing arm, or via any other means.

In various embodiments, the probing portion (e.g., angled probing portion) is made of stainless steel. In various embodiments, the angled probing portion is made of any suitable material.

In various embodiments, a long, narrow, hollow sheath fits over the probing arm. In various embodiments, if the probing arm is to bend, the sheath must bend as well in order to accommodate the probing arm inside it. In various embodiments, a sheath may be articulated. In various embodiments, a sheath may be capable of bending to mirror the bend in the probing arm within. For example, if the probing arm bends at 30 degrees, forming a corner 0.5 inches from the tip of the probing arm, then the sheath may similarly bend to form a 30 degree angle with a corner 0.5 inches from the tip of the sheath.

In various embodiments, the angle of bend in the sheath is controllable by the operator. The operator may use one or more actuators, buttons, etc., to adjust the bend in the sheath. The bend may be controlled via an adjustable hinge, a piezoelectric material that can expand or contract one side of the sheath (thereby causing it to bend), a fiber or pulley to preferentially pull one side of the sheath, or via any other means.

In various embodiments, the sheath interfaces to one or more actuators (e.g., on the main body of the device) (e.g., to finger-operated control actuators). The sheath can be bent at the location of the bend in the probing arm in order to conform to the shape of the probing arm, thereby forming an angle within the sheath. In various embodiments, an angle of the sheath is alterable in response to input from the one or more actuators.

In various embodiments, a sheath can come in pre-bent states. For example, a sheath may come with a 30-degree bend already built in. An operator may then, for example, choose an appropriate sheath at the start of a procedure based on a desired angle of bend. In various embodiments, multiple sheaths may be available. Each may have a different bend. In various embodiments, a sheath need not have a sharp bend, but may gradually curve such that the tip ultimately forms a particular angle with respect to the main axis of the device.

In various embodiments, sheaths may have different rigidities. In various embodiments, a sheath may be rigid. The sheath may be made of hard plastic or other rigid material. In various embodiments, the sheath may be semi-rigid. In various embodiments, the sheath may be flexible. Various embodiments contemplate that a sheath may be of any suitable rigidity or flexibility, and may be made of any appropriate material.

### Saline Channels

In various embodiments, the sheath includes one or more lumens for transmission of saline. In various embodiments, the lumen runs primarily parallel to the main axis of the sheath. I.e., the lumen primarily comprises a hollow channel inside the sheath. However, near the tip of the sheath, a channel may link the lumen to the outside environment. The channel may extend radially with respect to the axis of the sheath, or may otherwise be disposed perpendicular to the main trajectory of the lumen. In this way, saline may escape the sheath (or enter the sheath as the case may be) near the tip, but not quite at the tip. This may allow a greater surface area at the tip for the camera and/or light sources. This may also allow the axially disposed lumens to be used as anchor points for a cap that goes on the tip of the sheath.

Whereas the aforementioned lumens were described with respect to transmission of saline, it will be appreciated that various embodiments contemplate that such lumens or similar lumens could be used for transmission of other fluids or objects besides saline.

### Cap

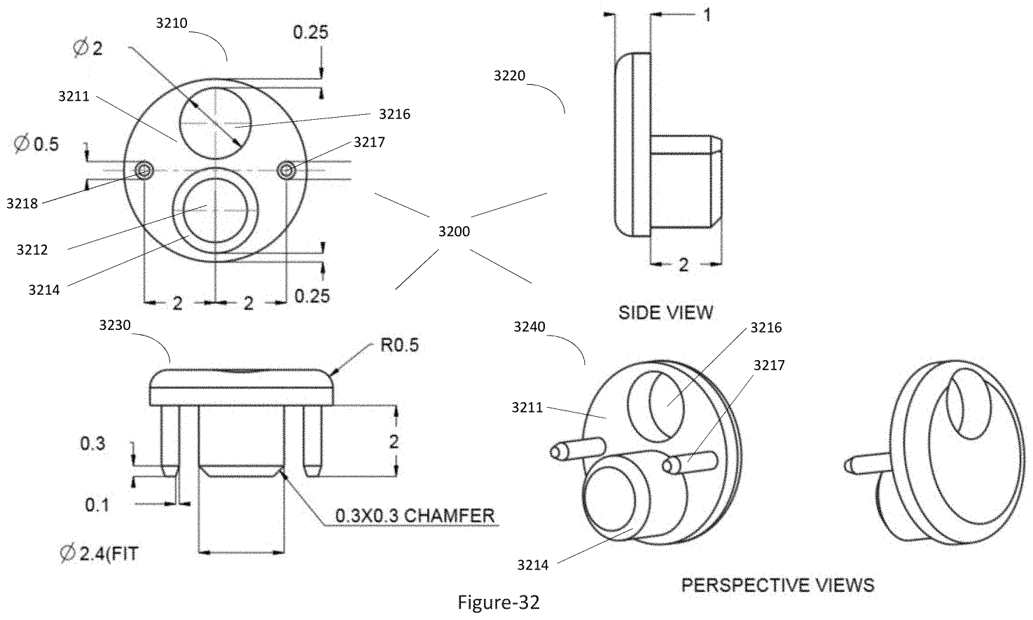

With reference to FIG. 32, a cap 3200 is described according to various embodiments. The cap may fit at the tip of a sheath. Various views of the cap 3200 are provided, including a front view 3210, side view 3220, top view 3230, and perspective views 3240. All measurements, captions, etc. depicted in FIG. 32 (and in other figures) shall be understood to represent illustrative embodiments, and shall not indicate that the illustrative embodiments are the only embodiments contemplated. Indeed, other embodiments are contemplated.

In various embodiments, the long arm of a sheath may be constructed using a tube with one or more lumens, and a cap that goes over one end of the tube. The cap 3200 may serve one or more functions. The cap may close off a lumen of the tube that is designed to house the probing portion of the device itself. Thus, because of the cap, the probing portion of the endoscopic device will avoid contact with the surrounding environment.

In various embodiments, the tip of the probing portion of the endoscopic device may include a camera and/or a light source. Thus, in various embodiments, at the point 3212 where the cap covers the lumen designed to house the probing portion of the endoscopic device, the cap may be transparent, and may thus allow transmission of light through the cap. In various embodiments, surrounding the transparent region (or point where the tip of the endoscopic device will be) may be a protruding portion 3214 (e.g., taking the shape of a hollow cylinder), where such protruding portion may fit (e.g., snugly) into the lumen design to house the probing portion of the endoscopic device.

In various embodiments, the cap may have an opening 3216 to allow a lumen of the tube to open to the environment through the opening in the cap. The lumen may be intended for admission of forceps or other tools.

In various embodiments, the cap may have a protruding portion 3217 (e.g., knob) designed to fit into the opening of a lumen from the tube (e.g., of a lumen designed for saline solution). The protruding portion may thus serve to plug or close off the corresponding lumen. The protruding portion may also better secure the cap to the tube. In various embodiments, there may be a plurality of protruding portions, each meant to plug into a different opening to a respective lumen. It will be appreciated that various embodiments may include more or fewer protruding portions than the number depicted in FIG. 32.

In various embodiments, protruding portions from the cap that mechanically plug into openings in lumens may provide extra security for keeping the cap fixed to the tube. The cap may also be glued to the tube at one or more points, including along the surfaces of the protruding portions. Thus, the protruding portions may also provide extra contact surface area with which to affix and/or adhere the cap to the tube.

In various embodiments, a sheath of an endoscopic device comprises a tube with a first lumen for the transmission of saline solution, and a second lumen for the admission of an imaging portion of the endoscopic device, the tube having a first end at which each of the lumens opens. The sheath may further include a cap 3200 attached to the first end. The cap may comprise a substantially flat portion 3211 covering the first end of the tube. The flat portion may be a disk, with the same shape and size as a cross-section of the tube. Part of the flat portion may be a transparent material positioned over an opening to the second lumen. The cap may further comprise a first protruding portion 3217. The first protruding portion may be disposed substantially perpendicularly to the flat portion. It may be shaped to snugly insert into an opening of the first lumen. I.e., the first protruding portion may have the same outer contours as the inner contour of the first lumen, but may be only slightly smaller so it can fit inside.

In various embodiments, the cap further comprises a third protruding portion 3214 shaped like a hollow cylinder with an axis substantially perpendicular to the flat portion and with an outer diameter shaped to fit snugly within the second lumen. Since the third protruding portion has a hollow, the camera and/or light source can be inserted into the second lumen and through the hollow of the third protruding portion and thereby be placed flush with the cap.

In various embodiments, the cap is attached to the first end of the tube with glue. As will be appreciated, various embodiments contemplate other means of attachment, including bonding, taping, stapling, melting together, pinning together, or any other suitable means of attachment.

In various embodiments, the tube of the sheath further comprises a third lumen for the transmission of saline. The cap may further comprise a second protruding portion 3218 disposed substantially perpendicularly to the flat portion, shaped to snugly insert into an opening of the third lumen.

In various embodiments, the tube of the sheath further comprises a third lumen for the admission of forceps. In various embodiments, the cap has a hole in the flat portion with the hole positioned over the opening to the third lumen.

In various embodiments, the tube of the sheath further comprises a fourth lumen for the transmission of saline. In various embodiments, the cap further comprises a second protruding portion disposed substantially perpendicularly to the flat portion, shaped to snugly insert into an opening of the fourth lumen.

In various embodiments, a sheath includes a first hole running radially outward from the first lumen to the side of the tube, the first hole permitting saline solution to escape out the side of the tube to avoid blockage by the first protruding portion of the cap.

In various embodiments, the cap is made polycarbonate. In various embodiments, the cap is made partially of polycarbonate. In various embodiments, the cap may be made of any other suitable material or combination of materials.

FIG. 32 depicts cap 3200 according to some embodiments. In various embodiments, various components of the cap 3200 may be arranged in different ways. In various embodiments protruding portions (e.g., 3217 and 3218) may be closer to one another, further to one another, or in any other suitable relationship to one another. Similarly, in various embodiments, the transparent region 3216 may be in a different spatial arrangement with respect to one of the protruding portions. Indeed, various components of the cap may be arranged to correspond to the cross-section of a particular tube (e.g., multi-lumen tube) being used as part of the sheath. In various embodiments, the components of cap 3200 may have different measurements and/or dimensions than those depicted. In various embodiments, there may be more or fewer of any given component, as appropriate.

### Other Embodiments

Various embodiments contemplate that one or more electronic components may be associated with the probing portion 110. For example, an LED or other light source may be located at or near the tip 125. As another example, camera 155 may be located at or near the tip 125. Such embodiments may be realized, for example, if the price of electronic components is reduced to sufficiently inexpensive levels. Various embodiments contemplate that other electronic components, components, and/or modules could be associated with (e.g., attached to) the probing portion 105. In such cases, these components may be intended for disposal after a single use.
The following are embodiments, not claims:
A. A device comprising:

a self-contained sensor assembly comprising:
a chassis;
an image sensor contained within the chassis;
a light source contained within the chassis;
a processor contained within the chassis;
a storage device contained within the chassis;
a memory contained within the chassis; and
a power source contained within the chassis,

a probing portion separable from the sensor assembly, the probing portion comprising:
a shell forming a cavity for holding the sensor assembly within;
a long, narrow shaft emanating from the outside of the shell at a first end of the shaft, and terminating in a tip at a second end of the shaft, the shaft comprising:
   a cylindrical, semi-rigid structural portion; and
   a plurality of optical fibers running the length of the shaft within the structural portion,
A.12 The device of embodiment A in which the a self-contained sensor assembly further comprises:

an optical coupler for transmitting light between the sensor assembly and the plurality of optical fibers.
A.10 The device of embodiment A in which the memory stores computer instructions and the processor executes such instructions to:

receive raw image data from the image sensor;

generate encoded image data based on the raw image data; and

transmit the encoded image data to an external device.
A.11 The device of embodiment A in which the memory stores computer instructions and the processor executes such instructions to:

receive raw video data from the image sensor;

generate encoded video data based on the raw image data; and

transmit the encoded video data to an external device.
A.13 The device of embodiment A in which the processor executes instructions provided by an operator.
According to various embodiments, a device may have one or more ports and/or paths.
A.9 The device of embodiment A in which the structural portion of the shaft further comprises:

a first port branching from the shaft, the first port comprising a first cylindrical shell with an opening to the environment at a first end of the first shell; and

a first hollow path running along the length of the shaft, opening into the environment at the tip of the shaft, and interfacing to the first port at a second end of the shell, in which a continuous tunnel is thereby formed running from the first end of the shell to the tip.
A.9.1 The device of embodiment A.9 in which the first hollow path has a diameter of 0.5 mm.
A.9.2 The device of embodiment A.9 further comprising a second port branching from the shaft, the second port comprising a second cylindrical shell with an opening to the environment at a first end of the second shell.
A.9.2.1 The device of embodiment A.9 further comprising a third port branching from the shaft, the third port comprising a third cylindrical shell with an opening to the environment at a first end of the third shell.
A.7 The device of embodiment A in which the shaft is between 1 and 3 millimeters in diameter.
A.8 The device of embodiment A in which the shaft is between 6 and 12 centimeters in length.
A.4 The device of embodiment A further comprising a lid for covering an entrance to the cavity when the sensor assembly is within the cavity.
In various embodiments, a user may press buttons or other actuators on a sensor assembly even through a shell.
A.15 The device of embodiment A in which self-contained sensor assembly further comprises an actuator for receiving user input, and in which a portion of the shell comprises pliable material situated above the actuator in order to transmit pressure from the outside surface of the shell to the actuator underneath.
A.3 The device of embodiment A further comprising a lens at the tip of the shaft.
A.4 The device of embodiment A in which the tip includes a planar outer surface that forms an angle with respect to a long axis of the shaft.
A.4.1 The device of embodiment A in which the angle is 30 degrees.
A.4.1 The device of embodiment A in which the angle is 60 degrees.
A.1. The device of embodiment A in which the light source is a light-emitting diode.
A.x. The device of embodiment A in which the power source is a battery.
A.x. The device of embodiment A in which the power source is an adapter connected to an electrical grid.
A.2 The device of embodiment A in which the probing portion is made from plastic.
A.2′ The device of embodiment A in which the probing portion is made from one or more of: (a) plastic; (b) glass; (c) metal; and (d) rubber.
A.5 The device of embodiment A further comprising a backflow stopper encircling a portion of the shaft.
A.5.1 The device of embodiment A.5 in which the backflow stopper is slidable along the length of the shaft.
A.5.2 The device of embodiment A.5 in which the backflow stopper has one or more grooves encircling its outer perimeter.
A.6 The device of embodiment A further comprising a transmitter.
A.6.1 The device of embodiment A.6 in which the transmitter is operable to transmit data to an external device.
A.6.2 The device of embodiment A.6 in which the transmitter is operable to wirelessly transmit data to an external device.
C. A system comprising:

the device of embodiment A; and

a separate display operable to display image data received from the device.
In various embodiments, the probing portion can be rotated about the sensor assembly to switch views.
A.12 The device of embodiment A in which:

the chassis of the sensor assembly has circular cross-sections perpendicular to a central axis;

the cavity formed by the shell of the probing portion has a complementary shape to that of the chassis; and

the sensor assembly is rotatable within the shell of the probing portion so as to rotate relative to the probing portion.
In various embodiments, the probing portion need not plug directly into the sensor assembly. In various embodiments, they are not directly connected. In various embodiments, one can rotate with respect to the other.
In various embodiments, no electronics are contained in the disposable portion.
A.12 The device of embodiment A in which the probing portion comprises solely electrically insulating material.
A.12′ The device of embodiment A in which the probing portion comprises solely electrically inactive components.
Various embodiments include a backflow stopper with a barrier.
D. A device for controlling fluid leakage during an endoscopic procedure, the device comprising:

a structural portion that is substantially cylindrically symmetrical about a central axis, the structural portion having a cross sectional profile that substantially increases in outer diameter over at least a portion of a length of the central axis;

a substantially hollow channel running along the central axis through the structural portion, the hollow channel permitting the admittance of a probing portion of an endoscopic device; and

a barrier closing off at least a portion of the hollow channel, the barrier comprising a material that is penetrable by the probing portion of the endoscopic device.
D.1 The device of embodiment D in which the barrier comprises silicone membrane.
D.2 The device of embodiment D in which the barrier is operable to seal around the outside of the probing portion after penetration by the probing portion.
Various embodiments include a backflow stopper with serrated sides.
E. A device for controlling fluid leakage during an endoscopic procedure, the device comprising:

a structural portion that is substantially cylindrically symmetrical about a central axis, the structural portion having a grooved outer surface with concentric grooves forming circles about the central axis;

a substantially hollow channel running along the central axis through the structural portion, the hollow channel permitting the admittance of a probing portion of an endoscopic device.
E.1 The device of embodiment E in which each of the concentric grooves has a peak and a trough, and in which peaks of successive grooves increase in diameter over at least a portion of a length of the central axis.
E.2 The device of embodiment E in which the grooves are concentric and non-intersecting.
Various embodiments include a backflow stopper slidable along sheath/probing portion.
F. An endoscope comprising:

a long, narrow probing arm;

a structural portion that is substantially cylindrically symmetrical about a central axis, the structural portion having:
a cross sectional profile that substantially increases in outer diameter over at least a portion of a length of the central axis; and
a substantially hollow channel running along the central axis through the structural portion, wherein the probing arm runs through the hollow channel,

in which the structural portion may be slid forwards or backwards along the probing arm.
F.1 The endoscope of embodiment F in which the structural portion maintains a substantially water-tight seal with the probing arm even as the structural portion is slid forwards or backwards along the probing arm.
F.2 The endoscope of embodiment F in which the probing arm comprises an outer sheath and an inner sensory portion, and in which, in sliding forward or backwards along the probing arm, the structural portion slides forward or backwards along the outer sheath.
A device according to various embodiments may be completely enclosed within a sheath.
G. An endoscope comprising:

a handheld body comprising electronics;

a long, narrow probing arm with a first end attached to the handheld body, and a second end having an imaging device;

a first sheath section comprising a long, narrow, hollow portion that fits over the probing arm;

a second sheath section comprising a hollow portion that fits over the handheld body; and

a snap mechanism for connecting the first sheath section and the second sheath section with a watertight seal, thereby fully isolating the handheld body and probing arm from any surroundings.
In various embodiments, the amount of bend in a sheath may be controllable (e.g., by an operator).
H. An endoscope comprising:

a handheld body comprising electronics and one or more finger-operated control actuators;

a long, narrow probing arm with a first end attached to the handheld body, and a second end having an imaging device, in which the probing arm is substantially straight but for a single bend which causes the second end to form a first angle with respect to the first end;

a long, narrow, hollow sheath that fits over the probing arm and interfaces to a first of the finger-operated control actuators, wherein the sheath can be bent at the location of the single bend in order to conform to the shape of the probing arm, thereby forming a second angle within the sheath;

in which the second angle of the sheath is alterable in response to input from the first of the finger-operated actuators.
I. A sheath of an endoscopic device, the sheath comprising:

a tube with a first lumen for the transmission of saline solution, and a second lumen for the admission of an imaging portion of the endoscopic device, the tube having a first end at which each of the lumens opens; and

a cap attached to the first end, the cap comprising:
a substantially flat portion covering the first end of the tube, wherein part of the flat portion is a transparent material positioned over an opening to the second lumen; and
a first protruding portion disposed substantially perpendicularly to the flat portion, shaped to snugly insert into an opening of the first lumen.
I.x The sheath of embodiment I in which the cap further comprises a third protruding portion shaped like a hollow cylinder with an axis substantially perpendicular to the flat portion and with an outer diameter shaped to fit snugly within the second lumen.
I.0 The sheath of embodiment I in which the cap is attached to the first end of the tube with glue.
I.1 The sheath of embodiment I in which the tube further comprises a third lumen for the transmission of saline, and in which the cap further comprises a second protruding portion disposed substantially perpendicularly to the flat portion, shaped to snugly insert into an opening of the third lumen.
I.2 The sheath of embodiment I in which the tube further comprises a third lumen for the admission of forceps, and in which the cap has a hole in the flat portion with the hole positioned over the opening to the third lumen.
I.2.1 The sheath of embodiment I.2 in which the tube further comprises a fourth lumen for the transmission of saline, and in which the cap further comprises a second protruding portion disposed substantially perpendicularly to the flat portion, shaped to snugly insert into an opening of the fourth lumen.
I.4 The sheath of embodiment I in which a first hole runs radially outward from the first lumen to the side of the tube, the first hole permitting saline solution to escape out the side of the tube to avoid blockage by the first protruding portion of the cap.
J. A charging dock for an endoscopic device having two detachable battery packs, the charging dock comprising:

a power brick with a connector to grid power;

a multi-component holster including:
a first cavity with a shape that is complementary to each of the detachable battery packs, the first cavity including an electrical interface to the power brick; and

a second cavity with a shape that is complementary to the endoscopic device with one of the two detachable battery packs still attached, the second cavity including an electrical interface to the power brick.

The aforementioned represent some embodiments, and it will be appreciated that embodiments not explicitly described are nevertheless contemplated, including embodiments falling within the spirit and scope of the aforementioned.

Applicants claim:

1. A device for controlling fluid leakage during an endoscopic procedure, the device comprising:
a structural portion that is substantially cylindrically symmetrical about a central axis, the structural portion having a cross sectional profile that substantially increases in outer diameter over at least a portion of a length of the central axis;
a substantially hollow channel running along the central axis through the structural portion, the hollow channel permitting the admittance of a probing portion of an endoscopic device; and
a barrier closing off at least a portion of the hollow channel, the barrier comprising a material that is penetrable by the probing portion of the endoscopic device.

2. The device of claim 1 in which the barrier comprises silicone membrane.

3. The device of claim 1 in which the barrier is operable to seal around the outside of the probing portion after penetration by the probing portion.

4. A device for controlling fluid leakage during an endoscopic procedure, the device comprising:
a structural portion that is substantially cylindrically symmetrical about a central axis, the structural portion having a grooved outer surface with concentric grooves forming circles about the central axis;
a substantially hollow channel running along the central axis through the structural portion, the hollow channel permitting the admittance of a probing portion of an endoscopic device.

5. The device of claim 4 in which each of the concentric grooves has a peak and a trough, and in which peaks of successive grooves increase in diameter over at least a portion of a length of the central axis.

6. The device of claim 4 in which the grooves are concentric and non-intersecting.
